SEQUENCE LISTING (51 sequences)

SEQ ID NO: 1 - protein, 198 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSRSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 2 - protein, 198 aa, Homo sapiens [sequence omitted]

SEQ ID NO: 3 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 4 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 5 - protein, 198 aa, Rattus norvegicus
MDSLLMKQKKFLYHFKNVRWAKGRHETYLCYVVKRRDSATSFSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENHERTFKAWEGLHENSVRLTRQLRRILLPLYEVDDLRDAFRILGL

SEQ ID NO: 6 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 7 - protein, 198 aa, Gallus gallus
MDSLLMKRKLFLYNFKNLRWAKGRRETYLCYVVKRRDSATSCSLDFGYLRNKMGCHVEVLFLRYISAWDLDPGRCYRITWFTSWSPCYDCARHVADFLRAYPNLTLRIFTARLYFCEDRKAEPEGLRRLHRAGAQIAIMTFKDFFYCWNTFVENREKTFKAWEGLHENSVHLSRKLRRILLPLYEVDDLRDAFKTLGL

SEQ ID NO: 8 - protein, 198 aa, Sus scrofa
MDSLLMKQRQFLYQFKNVRWAKGRHETYLCYVVKRRDSATSFSLDFGHLRNKSGCHVELLFLRYISDWDLDPGRCYRVTWFTSWSPCYDCARHVANFLRGNPNLSLRIFTARLYFCDGYKAEPEGLRRLHRAGVQIAIMTFKDYFYCWNTFVENRERSFKAWEGLHENSVRLTRQLRRILLPLYEVDDLRDAFRTLGL

SEQ ID NO: 9 - protein, 198 aa, Pan troglodytes
MDSLLMNRKKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 10 - protein, 198 aa, Macaca mulatta
MDSLLMNRRKFLYHFKNVRWAKGRHETYLCYVVKRRDSATSFSLDFGHLRNKSGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENRERTFKAWEGLHENSVRLSRQLRRILLPLYEVDDLRDAFRTLGL

SEQ ID NO: 11 - protein, 237 aa, Equus caballus
MSEVLTWAVPDAVPPVPNLLIHTAQEENDLCSFIFLPPLIHSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENRERTFKAWEGLHENSVRLSRQLRRILLPLYEVDDLRDAFRTLGL

SEQ ID NO: 12 - protein, 200 aa, Xenopus sp.
MTMDSMLLKRNKFIYHYKNLRWARGRHETYLCYIVKRRYSSVSCALDFGYLRNRNGCHAEMLFLRYLSIWVGHDPHRNYRVTWFSSWSPCYDCAKRTLEFLKGHPNFSLRIFSARLYFCEERNAEPEGLRKLQKAGVRLSVMSYKDYFYCWNTFVETRESGFEAWDGLHENSVRLARKLRRILQPPYDMEDLREVFVLLG

SEQ ID NO: 13 - protein, 204 aa, Takifugu rubripes
MLLPRKKFIYHYKNVRWARGRHETYLCFVVKRRVGPDTLTFDFGHLRNRSGCHVELLFLRYLGALCPGLWGYGAAGEKRLSYSVTWFCSWSPCVNCSIQLCQFLNNTPNLRLRIFVSRLYFCDLEDSLEREGLRMLTKAGVRISVMSYKDYFYCWQKFVDCKKSNFKAWEELHQNSVRLTRKLNRILQAWDLEDLRDALKLLGF

SEQ ID NO: 14 - protein, 210 aa, Danio rerio
MICKLDSVLMTQKKFIFHYKNVRWARGRHETYLCFVVKRRIGPDSLSFDFGHLRNRSGCHVELLFLRHLGALCPGLSASSVDGARLCYSVTWFCSWSPCSKCAQQLAHFLSQTPNLRLRIFVSRLYFCDEEDSVEREGLRHLKRAGVQISVMTYKDFFYCWQTFVARRERSFKAWDGLHENSVRLVRKLNRILQPCETEDLRDVFALLGL

SEQ ID NO: 15 - DNA, 645 bp, Artificial Sequence
  Synthetic
atggactacaaagatgacgatgataaaggtccaaagaagaagagaaaggtagactctctcctcatgaagcagagaaagtttctctaccacttcaagaacgtcagatgggccaaggggagacatgagacctatctctgttacgtcgtcaagaggagagactcagccacctctttctccctcgactttgggcatctccggaacaagtctgggtgtcatgtcgaactcctcttcctccgctatatctcagactgggacctcgaccccgggagatgctatagagtcacttggtttacctcttggtccccctgttatgactgcgccagacatgtcgccgacttcctcagggggtatcccaatctctccctccgcatattcgccgcccgactctatttttgtgaggacaggaaagccgagcccgaggggctcaggagactccaccgggccggggtccagatcgccatcatgacatttaaggactatttctattgttggaatacatttgtcgagaatcgggagaagactttcaaagcctgggaggggctccatgagaactctgtcagactctctaggcagctcaggagaatcctcctccccctctatgaggtcgaagaactcagagaagccttccggatcctcggggcttga

SEQ ID NO: 16 - DNA, 480 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctcagggagtgcatccgccccaacccttttcccgctagcaccc

SEQ ID NO: 17 - DNA, 467 bp, Artificial Sequence
  Synthetic
atggactggacctggaggttcctctttgtggtggcagcagctacaggtgtccagtcccaggtgcagctggttcagtctggggctgaggtgaactgggtgcagctggttcagtctggggctgaggtgaagaagcctgggtcctcggtgaaggtctcctgcaagacttctggaggcaccttcagcaactatgctatcagctgggtgcgacaggcccctggacaagggcttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaaccctggtca

SEQ ID NO: 18 - DNA, 479 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggaggcaccttcagcaactatgctatcagcttcagcaactatgctatcagttgggtgcgacaggcccctggacaagggcttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctcagggagtgcatccgcccc

SEQ ID NO: 19 - DNA, 469 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggagacaccttcagcaactatgctactggagacaccttcagcaactatgctatcagctgggtgcgacaggcccctggacaagggtttgagtggatgggagggatcatccctatctttggtatagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctcag

SEQ ID NO: 20 - DNA, 462 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggaggcaccttcagcaactatgctatcaccttcagcaactatgctatcagctgggtgcgacaggcccctggacaagggtttgagtggatgggagggatcatccctatctttgctacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctca

SEQ ID NO: 21 - DNA, 476 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcggcttctggaggcaccttcagcaactatgctatcagctgggtgcgacaggcccctggacaagggcttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctcagggagtgcatccgccccaacccttttcccgct

SEQ ID NO: 22 - DNA, 474 bp, Artificial Sequence
  Synthetic
atggactggacctggaggttcctctgtgtggtggcagcagctacaggtgtccagtaccaggtgcagctggttcagtctggggccgaggtgaaganncctgggtcctcggtgaaggtctcctgcaaggcttctggaggcaccttcagcaactatgctatcaccttcagcaactatgctatcagctgggtgcgacaggcccctggacaagggtttgagtggatgggagggatcctccctatctttggtacagcaaactacgcacagaagttccaggacagagtcacgattcccggaggagaatccacgagcacagtctacatggaactaagcagcctgagatcagaggacacggccgtgtattactgtgcgagaggtccggagtattatgattncgtttgggggagttatcgttataaatactttgactactggggccagggaaccctggtcaccgtctcctcagggagtgcatct

SEQ ID NO: 23 - protein, 160 aa, Artificial Sequence
  Synthetic
MDWTWRFLFVVAAATGVQSQVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGPEYYDYVWGSYRYNYFDYWGQGTLVTVSSGSASAPTLFPLAP

SEQ ID NO: 24 - protein, 152 aa, Artificial Sequence
  Synthetic
MDWTWRFLFVVAAATGVQSQVQLVQSGAEVNWVQLVQSGAEVKKPGSSVKVSCKTSGGTFSNYAISWVRQAPGQGLEWMGGIIPIFGTANYAQKFQGRVTITADESTSTAYMELSSLRSEDTAVYYCARGPEYYDYVWGSYRYNYFDYWGQG

SEQ ID NO: 25 - protein, 159 aa, Artificial Sequence
  Synthetic
MDWTWRFLFVVAAATGVQSQVQLVQSGAEVKKPGSSVKVSCKASGGTFSNYAISFSNYAISWVRQAPGQGLEWMGGIIPIFGTANYAQKFQGRVTITADESTSTAYMELSSLRSEDTAVYYCARGPEYYDYVWGSYRYNYFDYWGQGTLVTVSSGSASA

SEQ ID NO: 26 - protein, 156 aa, Artificial Sequence
  Synthetic
MDWTWRFLFVVAAATGVQSQVQLVQSGAEVKKPGSSVKVSCKASGDTFSNYATGDTFSNYAISWVRQAPGQGFEWMGGIIPIFGIANYAQKFQGRVTITADESTSTAYMELSSLRSEDTAVYYCARGPEYYDYVWGSYRYNYFDYWGQGTLVTVSS

SEQ ID NO: 27 - protein, 154 aa, Artificial Sequence
  Synthetic
MDWTWRFLFVVAAATGVQSQVQLVQSGAEVKKPGSSVKVSCKASGGTFSNYAITFSNYAISWVRQAPGQGFEWMGGIIPIFATANYAQKFQGRVTITADESTSTAYMELSSLRSEDTAVYYCARGPEYYDYVWGSYRYNYFDYWGQGTLVTVSS

SEQ ID NO: 28 - protein, 158 aa, Artificial Sequence
  Synthetic
MDWTWRFLFVVAAATGVQSQVQLVQSGAEVKKPGSSVKVSCKAASGGTFSNYAISWVRQAPGQGLEWMGGIIPIFGTANYAQKFQGRVTITADESTSTAYMELSSLRSEDTAVYYCARGPEYYDYVWGSYRYNYFDYWGQGTLVTVSSGSASAPTLFP

SEQ ID NO: 29 - DNA, 479 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacagattactacatacactactggtacttcgatctctggggccgtggcaccctggtcactgtctcctcagcctccaccaagggcccatcggtcttcccgctagcaccctc

SEQ ID NO: 30 - DNA, 480 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacaagagattactatgatatacttacagattactacatacactactggtacttcgatctctggggccgtggcaccctggtcactgtctcctcagcctccaccaagggccca

SEQ ID NO: 31 - protein, 159 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCARDYYDILTDYYIHYWYFDLWGRGTLVTVSSASTKGPSVFPLAP

SEQ ID NO: 32 - protein, 167 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCARDYYDILTRDYYDILTDYYIHYWYFDLWGRGTLVTVSSASTKGPSVFPLAP

SEQ ID NO: 33 - DNA, 416 bp, Artificial Sequence
  Synthetic
atggaattggggctgatgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacagattactacatacactactggtacttcgatctctggggccg

SEQ ID NO: 34 - DNA, 420 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcatctggattcacctttagttctcactggatgagctgggtccgccaggctccagggaaagggctggagtgggtggccagcataaaggaagatggaagtgagaaatattatgtggactctgtgaagggccgattcaccatctccagagacaacgccaagaactcactgtatctgcaaatgaacagcctgagagctgaggacacggctgtgtattactgtgcaagagattactatgatatacttacagattactacatacactactggtacttcgatctctggggccgtggcaccctg

SEQ ID NO: 35 - DNA, 416 bp, Artificial Sequence
  Synthetic
atggaattggggctgagctgggttttccttgttgctattttagaaggtgtccagtgtgaggtggtgcagttggtggagtctgggggaggcttggtccagcctggggggtccctgagactctcctgtgcagcctctggattcacctttagtgattattggatggattgggtccgccaggctccagggaaagggctggagtgggtggccagcataagggaagatggaagtgagaaatattatgtggactctgtgaagggccgattcaccatctccagagacaacgccaagaactcactgtatctgcaaatgaacagcctgagagctgaggacacggctgtgtattactgtgcaagagattactatgatatacttacagattactacatacactactggtacttcgatctctggggccg

SEQ ID NO: 36 - DNA, 482 bp, Artificial Sequence
  Synthetic
atgaacaagttgctgtgctgcgcgctcgtgtttctggacatctccattaagtggaccacccaggacgtgcagcttcaggagtcaggacctagcctcgtgaaaccttctcagactctgtccctcacctgttctgtcactggcgactccatcaccagtgattactggagctggatccggaaattcccagggaatagacttgagtacatggggtacgtaagctacagtggtagcacttactacaatccatctctcaaaagtcgaatctccatcacccgagacacatccaagaaccagtactacctggatttgaattctgtgactactgaggacacagccacatattactgtgcaaactgggacggtgattactggggccaagggactctggtcactgtctctgcagccaaaacgacacccccatctgtctatccactggccaagggcgaattctgcagatatccatcacactggcggccgctcga

SEQ ID NO: 37 - DNA, 497 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcaagatacctaaagataagtgcactcaaggctgattttgcaaagggctactacgctatggacgtctggggccaagggaccacggtcaccgtctcctccgcttccaccaagggcccatccgtcttcccgctagcaccctcctccaagagcac

SEQ ID NO: 38 - DNA, 363 bp, Artificial Sequence
  Synthetic
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaaccattagtaccggcggtggttacacatacttccccgactccgtgaagggccggttcaccatctccagagacaattccaagaacacgctgtatctgcaaatgaacagcctgagagccgaggacacggccgtatattactgtgcgagacagggcgacttcggcgactggtacttcgatgtgtggggcgcaggcaccaccgtcaccgtctcctcagcc

SEQ ID NO: 39 - DNA, 31 bp, Artificial Sequence
  Synthetic
atgaagcagagagagtttctctaccacttca

SEQ ID NO: 40 - DNA, 14 bp, Artificial Sequence
  Synthetic
tcagggggtatccc

SEQ ID NO: 41 - DNA, 59 bp, Artificial Sequence
  Synthetic
ggggtatcccaatctctccctccgcatattcgccgcccgactctatttttgtgaggaca

SEQ ID NO: 42 - DNA, 19 bp, Artificial Sequence
  Synthetic
cccgactctatttttgtga

SEQ ID NO: 43 - DNA, 21 bp, Artificial Sequence
  Synthetic
tatttttgtgaggacaggaaa

SEQ ID NO: 44 - DNA, 21 bp, Artificial Sequence
  Synthetic
tatttttgtgaggacaggaaa

SEQ ID NO: 45 - DNA, 13 bp, Artificial Sequence
  Synthetic
cctcgaccgggcc

SEQ ID NO: 46 - DNA, 10 bp, Artificial Sequence
  Synthetic
ttcaaagcct

SEQ ID NO: 47 - DNA, 15 bp, Artificial Sequence
  Synthetic
tcacctgttctgtca

SEQ ID NO: 48 - DNA, 13 bp, Artificial Sequence
  Synthetic
caaactgggacgg

SEQ ID NO: 49 - DNA, 11 bp, Artificial Sequence
  Synthetic
aaggcttctgg

SEQ ID NO: 50 - DNA, 480 bp, Artificial Sequence
  Synthetic
atggactggacctggaggttcctctttgtggtggcagcagctacaggtgtccagtcccaggtgcagctggttcagtctggggctgaggtgaagaagcctgggtcctcggtgaaggtctcctgcaaggcttctggaggcaccttcagcaactatgctatcagctgggtgcgacaggcccctggacaagggtttgagtggatgggagggatcatccctatctttggtacagcaaactacgcacagaagttccagggcagagtcacgattaccgctgacgaatccacgagcacagcctacatggaactaagcagcctgagatcagaggacacggccgtgtattactgtgcgagaggtccggagtattatgattacgtttgggggagttatcgttataactactttgactactggggccagggaaccctggtcaccgtctcctcagggagtgcatccgccccaacccttttcccgctagcaccc

SEQ ID NO: 51 - DNA, 420 bp, Artificial Sequence
  Synthetic
atggaattggggctgagctgggttttccttgttgctattttagaaggtgtccagtgtgaggtgcagttggtggggtctgggggaggcttggtccagcctggggggtccctgagactctcctgtgcagcctctggattcacctttagtaaccattggatggactgggtccgccagactccagggaaagggctggagtgggtggccctaaaggaagatggaagtgagaaatactatgtgggctctgtgaaggtccgattcaccatctccagagacaacgccaagaactcactgtatctgcaaatgaacagcctgagagtcgaggacacggctgtgtattactgtgtgagagattactatgatatacttacagattactacatacactactggtacttcgatctctggggccgtggcaccctg

(19) United States
(12) Patent Application Publication
(10) Pub. No.: US 2013/0102031 A1
(43) Pub. Date: Apr. 25, 2013

(54) USE OF SOMATIC HYPERMUTATION TO CREATE INSERTION AND DELETION MUTATIONS IN VITRO

(71) Applicant: AnaptysBio, Inc., San Diego, CA (US)

(72) Inventors: David J. King, Encinitas, CA (US); Peter Bowers, San Diego, CA (US); Robert A. Horlick, San Diego, CA (US)

(73) Assignee: ANAPTYSBIO, INC., San Diego, CA (US)

(21) Appl. No.: 13/655,793

(22) Filed: Oct. 19, 2012

Related U.S. Application Data
(60) Provisional application No. 61/551,030, filed on Oct. 25, 2011.

(51) Int. Cl. C12P 19/34; C12P 21/00; C12N 15/13

(52) U.S. Cl. 435/69.6; 435/91.1; 536/23.53

(57) ABSTRACT

The invention is directed to an in vitro method for introducing one or more insertion mutations and/or one or more deletion mutations in a nucleic acid sequence encoding a polypeptide. The method comprises contacting a cell in vitro with a nucleic acid sequence, and expressing Activation Induced Cytidine Deaminase (AID) in the cell. The invention also is directed to a method of producing an immunoglobulin.

### CROSS-REFERENCE TO RELATED APPLICATIONS

This patent application claims the benefit of U.S. Provisional Patent Application No. 61/551,130, filed Oct. 25, 2011, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 52,364 Byte ASCII (Text) file named “711272_ST25.TXT,” created on Oct. 19, 2012.

### BACKGROUND OF THE INVENTION

Natural mechanisms for generating antibody diversity (e.g., affinity maturation) exploit the process of somatic hypermutation (SHM) to trigger the evolution of immunoglobulin variable regions, thereby rapidly generating the secondary antibody repertoire associated with a humoral response. In vivo, SHM is a highly efficient process, which is capable of rapidly exploring productive folding structures and evolving high affinity antibodies in a manner that represents the natural process for antibody optimization. Somatic hypermutation of immunoglobulin genes during affinity maturation typically involves the generation of point mutations that are initiated by, or otherwise dependent on, the action of activation-induced cytidine deaminase (AID). In addition to point mutations, somatic insertions and deletions of nucleic acids are a rare but important feature of affinity maturation of antibodies in vivo (see, e.g., Sale and Neuberger, Immunity, 9: 859-869 (1998), Miura et al., Molecular Medicine, 9(5-8): 166-174 (2003), Wilson et al., J. Exp. Med., 187: 59-70 (1996), and Ichikawa et al., J. Immunology, 177: 355-361 (2006)). These somatic nucleic acid insertions and deletions appear to be generated by an AID-dependent mechanism that is related to SHM (see, e.g., Krause et al., mBio, 2(1): e00345-10 (2011), Wu et al., J. Clin. Immunology, 23(4): 235-246 (2003), and Reason and Zhou, Biology Direct, 1: 24 (2006)).

There has been significant interest to try to replicate SHM in vitro to create a simple, robust process that would be capable of mimicking the natural processes of affinity maturation directly within a mammalian cellular context to select and evolve antibodies that are immunogenically tolerated and highly expressed in mammalian cells (see, e.g., Cumbers et al., Nat. Biotechnol., 20(11): 1129-1134 (2002), Wang et al., Prot. Eng. Des. Sel., 17(9): 569-664 (2004), Wang et al., Proc. Natl. Acad. Sci. USA., 101(48): 16745-16749 (2004), Ruckerl et al., Mol. Immunol., 43 (10): 1645-1652 (2006), Todo et al., J. Biosci. Bioeng., 102(5): 478-81 (2006), and Arakawa et al., Nucleic Acids Res., 36(1): e1 (2008)). However, the generation of nucleic acid sequence insertions or deletions in immunoglobulin genes to effect improved binding affinity is an important feature of in vivo affinity maturation that has not been demonstrated in an in vitro system.

There remains a need for alternative and improved in vitro methods for generating nucleic acid sequence insertions and deletions within a gene, particularly an immunoglobulin gene, which results in the production of a polypeptide having an improved function. This invention provides such a method.

### BRIEF SUMMARY OF THE INVENTION

The invention provides a method for introducing one or more insertion mutations and/or one or more deletion mutations in a nucleic acid sequence encoding a polypeptide. The method comprises (a) providing a cell in vitro that expresses or can be induced to express Activation Induced Cytidine Deaminase (AID), (b) contacting the cell in vitro with a nucleic acid sequence that encodes a polypeptide, and (c) expressing AID in the cell, whereupon one or more insertion mutations and/or one or more deletion mutations are introduced in the nucleic acid sequence encoding the polypeptide.

The invention also provides a method of producing an immunoglobulin. The method comprises (a) providing a cell in vitro that expresses or can be induced to express AID, (b) contacting the cell in vitro with a nucleic acid sequence encoding an immunoglobulin heavy chain polypeptide that contains one or more insertion mutations and/or one or more deletion mutations and a nucleic acid sequence encoding an immunoglobulin light chain polypeptide that contains one or more insertion mutations and/or one or more deletion mutations, and (c) expressing AID in the cell, whereupon an immunoglobulin comprising a heavy chain polypeptide and a light chain polypeptide is produced in the cell.

### BRIEF DESCRIPTION OF THE SEVERAL VIEWS OF THE DRAWING(S)

FIG. 1 is an alignment of a portion of the nucleic acid sequences (i.e., SEQ ID NO: 16, SEQ ID NO: 17, SEQ ID NO: 18, SEQ ID NO: 19, SEQ ID NO: 21, and SEQ ID NO: 22) encoding heavy chain variable regions of an anti-NGF antibody described in Example 1. The amino acid sequence for each of the nucleic acid sequences is shown above their respective nucleic acid sequence. Regions of the original nucleic acid sequence are in italics, the CDR1 is boxed and in bold text, and duplicated sequences are indicated by underlining.

FIG. 2 is an alignment of the full-length nucleic acid sequences encoding heavy chain variable regions of an anti-NGF antibody described in Example 1 (i.e., SEQ ID NOs: 16-21).

FIG. 3 is an alignment of the full-length amino acid sequences of the heavy chain variable regions of an anti-NGF antibody described in Example 1 (i.e., SEQ ID NOs: 23-28).

FIG. 4 is a graph which depicts experimental data illustrating the size distribution of anti-NGF heavy chain variable regions described in Example 1.

FIGS. 5a-5f are graphs which depict experimental data illustrating the association and dissociation kinetic constants (“kₒₙ” and “k_{off}”) and steady-state affinity (KD) of the anti-NGF heavy chain variable region polypeptides encoded by SEQ ID NO: 16 (FIG. 5a), SEQ ID NO: 50 (FIG. 5b), SEQ ID NO: 21 (FIG. 5c), SEQ ID NO: 20 (FIG. 5d), SEQ ID NO: 18 (FIG. 5e), and SEQ ID NO: 19 (FIG. 5f).

FIG. 6 is a graph which depicts experimental data illustrating the IC₅₀ values of anti-NGF heavy chain variable region polypeptides encoded by SEQ ID NO: 16 and SEQ ID NO: 19, as determined by the HTRF assay described in Example 1.

FIG. 7 is an alignment of a portion of the nucleic acid sequences (i.e., SEQ ID NO: 29 and SEQ ID NO: 30) encoding anti-IL17a heavy chain variable regions described in Example 2. The amino acid sequence for each of the nucleic acid sequences is shown above its respective nucleic acid sequence. Regions of the original nucleic acid sequence are in italics, the CDR3 is boxed and in bold text, and duplicated sequences are indicated by underlining.

FIG. 8 is an alignment of the full-length nucleic acid sequences encoding heavy chain variable regions of an anti-IL17a antibody described in Example 2 (i.e., SEQ ID NOs: 29 and 30).

FIG. 9 is an alignment of the full-length amino acid sequences of the heavy chain variable regions of an anti-IL17a antibody described in Example 2 (i.e., SEQ ID NOs: 31 and 32).

FIGS. 10a and 10b are graphs which depict experimental data illustrating the antigen binding affinity of an anti-IL17a antibody comprising a heavy chain encoded by SEQ ID NO: 29 (FIG. 10a) and an anti-IL17a antibody comprising a heavy chain encoded by SEQ ID NO: 30 (FIG. 10b). The heavy chain polypeptide encoded by SEQ ID NO: 30 contains an insertion mutation in the CDR3.

FIG. 11 is a graph which depicts experimental data illustrating the size distribution of anti-IL17a heavy chain variable regions encoded by SEQ ID NO: 29 and SEQ ID NO: 30, described in Example 2.

FIG. 12 is a table which describes the location, type, and size of insertion and deletion mutations introduced in nucleic acid sequences encoding an anti-MS2 antibody heavy chain polypeptide prior to fluorescent activated cell sorting (FACS) selection as described in Example 4.

FIG. 13 is a table which describes the location, type, and size of insertion and deletion mutations introduced in nucleic acid sequences encoding an anti-MS2 antibody heavy chain polypeptide following fluorescent activated cell sorting (FACS) selection as described in Example 4.

### DETAILED DESCRIPTION OF THE INVENTION

The invention provides a method for introducing one or more insertion mutations and/or one or more deletion mutations in a nucleic acid sequence encoding a polypeptide.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acids or polynucleotides, though many other linkages are known in the art (e.g., phosphorothioates, boranophosphates, and the like). The nucleic acid sequence can be eukaryotic or prokaryotic in origin. Preferably, the nucleic acid sequence is eukaryotic in origin.

The nucleic acid sequence encodes a polypeptide (also referred to herein as a “protein”). The nucleic acid sequence can encode one or more polypeptides (e.g., 2 or more, 3 or more, 4 or more, or 5 or more polypeptides). In a preferred embodiment, however, the nucleic acid sequence encodes a single polypeptide. The polypeptide can be any suitable polypeptide, including, for example, surface proteins, intracellular proteins, membrane proteins, and secreted proteins from any unmodified or synthetic source. The polypeptide preferably is an immunoglobulin heavy chain polypeptide or portion thereof, an immunoglobulin light chain polypeptide or portion thereof, an enzyme, a receptor, a structural protein, a co-factor, an intrabody, a selectable marker, a toxin, a growth factor, or a peptide hormone.

The polypeptide can be any suitable enzyme, including enzymes associated with microbiological fermentation, metabolic pathway engineering, protein manufacture, bio-remediation, DNA repair, and plant growth and development (see, e.g., Olsen et al., Methods Mol. Biol., 230: 329-349 (2003); Turner, Trends Biotechnol., 21(11): 474-478 (2003); Zhao et al., Curr. Opin. Biotechnol., 13(2): 104-110 (2002); and Mastrobattista et al., Chem. Biol., 12(12): 1291-300 (2005)).

The polypeptide can be an antigen. An “antigen” is any molecule that induces an immune response in a mammal. An “immune response” can entail, for example, antibody production and/or the activation of immune effector cells (e.g., T-cells). An antigen in the context of the invention can comprise any subunit, fragment, or epitope of any proteinaceous or non-proteinaceous (e.g., carbohydrate or lipid) molecule which provokes an immune response in a mammal. By “epitope” is meant a sequence on an antigen that is recognized by an antibody or an antigen receptor. Epitopes also are referred to in the art as “antigenic determinants.”

In a preferred embodiment of the invention, the polypeptide is an antibody (also referred to herein as an “immunoglobulin”) or a portion thereof. For example, the polypeptide can be a whole antibody. A whole antibody consists of four polypeptides: two identical copies of a heavy (H) chain and two copies of a light (L) chain. Each of the heavy chains contains one N-terminal variable (VH) region and three C-terminal constant (CH1, CH2, and CH3) regions, and each light chain contains one N-terminal variable (VL) region and one C-terminal constant (CL) region. In a typical whole antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other. Preferably, the nucleic acid sequence encodes an immunoglobulin heavy chain polypeptide or an immunoglobulin light chain polypeptide, or fragments (e.g., immunogenic fragments) thereof. The light chains of antibodies can be assigned to one of two distinct types, either kappa (κ) or lambda (λ), based upon the amino acid sequences of their constant domains. Preferably, the light chain is a kappa light chain.

The variable regions of each pair of light and heavy chains form the antigen binding site of an antibody. The VH and VL regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding. The four framework regions (FWs or FRs) largely adopt a beta-sheet conformation, and the CDRs form loops connecting, and in some cases comprising part of, the beta-sheet structure.

The constant regions of the light and heavy chains are not directly involved in binding of the antibody to an antigen, but exhibit various effector functions, such as participation in antibody-dependent cellular toxicity via interactions with effector molecules and cells.

In one embodiment, the polypeptide can be a fragment of an antibody. The terms “fragment of an antibody,” “antibody fragment,” or “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). Examples of antibody fragments include, but are not limited to, (i) a Fab fragment, which is a monovalent fragment consisting of the VL, VH, CL, and CH1 domains, (ii) a F(ab′)2 fragment, which is a bivalent fragment comprising two Fab fragments linked by a disulfide bridge at the hinge region, and (iii) a Fv fragment consisting of the VL and VH domains of a single arm of an antibody.

In embodiments where the nucleic acid sequence encodes a fragment of an immunoglobulin heavy chain or light chain polypeptide, the fragment can be of any size so long as the fragment binds to, and preferably inhibits the activity of, a target antigen or epitope. In this respect, a fragment of the immunoglobulin heavy chain polypeptide desirably comprises between about 5 and 18 amino acids (e.g., about 5, 6, 7, 8, 9, 10, 11, 12, 13, 14, 15, 16, 17, 18, or a range defined by any two of the foregoing values). Similarly, a fragment of the immunoglobulin light chain polypeptide desirably comprises between about 5 and 18 amino acids (e.g., about 5, 6, 7, 8, 9, 10, 11, 12, 13, 14, 15, 16, 17, 18, or a range defined by any two of the foregoing values). When the nucleic acid sequence encodes an antibody or antibody fragment, the antibody or antibody fragment comprises a constant region (F_{c}) of any suitable class. Preferably, the antibody or antibody fragment comprises a constant region that is based upon a wild-type IgG1, IgG2, or IgG4 antibody, or variant thereof.

In another embodiment, the polypeptide can be a single chain antibody fragment. Examples of single chain antibody fragments include, but are not limited to, (i) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423 426 (1988); and Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH and VL polypeptide chains to generate a dimeric molecule having two functional antigen binding sites. Antibody fragments are known in the art and are described in more detail in, e.g., U.S. Patent Application Publication 2009/0093024 A1.

The polypeptide can also be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated VH and VL domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or induces phenotypic/functional knockout of the target protein by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to an epitope that is involved in protein-protein, protein-DNA, or protein-RNA interactions.

The nucleic acid sequence can encode a human antibody, a non-human antibody, or a chimeric antibody. By “chimeric” is meant an antibody or fragment thereof comprising both human and non-human regions. Non-human antibodies include antibodies isolated from any non-human animal, such as, for example, a rodent (e.g., a mouse or rat).

A nucleic acid sequence encoding a human antibody, a non-human antibody, or a chimeric antibody can be obtained by any means, including in vitro sources (e.g., a hybridoma or a cell line producing an antibody recombinantly) and in vivo sources (e.g., rodents). Methods for generating antibodies, and nucleic acid sequences encoding them, are known in the art and are described in, for example, see, e.g., Köhler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). In certain embodiments, a human antibody-encoding nucleic acid sequence or a chimeric antibody-encoding nucleic acid sequence can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE®, the Kirin TC MOUSE™, and the Kyowa Kirin KM-MOUSE® (see, e.g., Lonberg N., Nat. Biotechnol., 23(9): 1117-25 (2005), and Lonberg N., Handb. Exp. Pharmacol., 181: 69-97 (2008)).

Instead of an antibody or a fragment thereof, the polypeptide also can be an “alternative scaffold” or a fragment thereof. By “alternative scaffold” is meant a non-antibody polypeptide or polypeptide domain which displays an affinity and specificity towards an antigen of interest similar to that of an antibody. Exemplary alternative scaffolds include a β-sandwich domain such as from fibronectin (e.g., Adnectins), lipocalins (e.g., Anticalin), a Kunitz domain, thioredoxin (e.g., peptide aptamer), protein A (e.g., AFFIBODY® molecules), an ankyrin repeat (e.g., DARPins), γ-β-crystallin or ubiquitin (e.g., AFFLIN™ molecules), CTLD3 (e.g., Tetranectin), and multivalent complexes (e.g., ATRIMER™ molecules or SIMP™ molecules). Alternative scaffolds are described in, for example, Binz et al., Nat. Biotechnol., 23: 1257-1268 (2005); Skerra, Curr. Opin. Biotech., 18: 295-304 (2007); and U.S. Patent Application Publication 2009/0181855 A1.

In another embodiment, the polypeptide can be a conjugate of (1) an antibody, an alternative scaffold, or fragment thereof, and (2) a protein or non-protein moiety. For example, the polypeptide can be an antibody conjugated to a peptide, a fluorescent molecule, or a chemotherapeutic agent. The polypeptide also can be a fusion protein. Fusion proteins are generated by transcriptionally linking two or more nucleic acid sequences which code for separate proteins. Translation of the linked genes produces a single polypeptide with functional properties derived from each of the individual proteins. In the context of the invention, the nucleic acid sequence encodes a naturally-occurring fusion protein (e.g., an antibody protein or the bcr-abl fusion protein), or, using recombinant DNA techniques known in the art, the nucleic acid sequence can be generated to encode a synthetic fusion protein. For example, a nucleic acid sequence encoding a peptide tag can be ligated to a second nucleic acid sequence encoding a polypeptide of interest to facilitate protein purification and/or identification. Suitable peptide tags include, for example, a glutathione-S-transferase (GST) protein, a FLAG peptide, or a polyhistidine (HIS) tag. Fc fusion proteins are another type of synthetic fusion protein that can be used in the invention. Fc fusion proteins contain a soluble antibody constant fragment (Fc). Soluble Fc fusion proteins can be used as reagents for several in vitro and in vivo applications, including, but not limited to, immunotherapy, flow cytometry, immunohistochemistry, and in vitro activity assays. Fc fusion proteins are described in, for example, Flanagan et al., “Soluble Fc Fusion Proteins for Biomedical Research,” in Monoclonal Antibodies: Methods and Protocols (Methods in Molecular Biology), M. Albitar (ed.), Humana Press, Inc., pp. 33-52 (2008). The fusion protein can be used for therapeutic or diagnostic purposes. For example, a therapeutic fusion protein can be generated in which one portion of the fusion protein is capable of directing the fusion protein to a specific cell or tissue, while the other portion of the fusion protein is a biologically active protein or peptide (also referred to in the art as a “payload”), such as an antibody or a cytotoxic protein.

In certain embodiments of the invention, the nucleic acid sequence can be modified as compared to a corresponding wild-type nucleic acid sequence to increase or decrease the density of somatic hypermutation (SHM) cold spots and/or SHM hot spots so as to increase or decrease the susceptibility of the nucleic acid sequence to SHM. As used herein, the term “SHM hot spot” or “hot spot” refers to a polynucleotide sequence, or motif, of 3-6 nucleotides that exhibits an increased tendency to undergo somatic hypermutation, as determined via a statistical analysis of SHM mutations in antibody genes. Likewise, as used herein, a “SHM cold spot” or “cold spot” refers to a polynucleotide sequence, or motif, of 3-6 nucleotides that exhibits a decreased tendency to undergo somatic hypermutation, as determined via a statistical analysis of SHM mutations in antibody genes. A relative ranking of various motifs for SHM as well as canonical hot spots and cold spots in antibody genes are described in U.S. Patent Application Publication 2009/0075378 A1 and International Patent Application Publication WO 2008/103475, and the statistical analysis can be extrapolated to an analysis of SHM mutations in non-antibody genes as described therein.

In some aspects, the number of hotspots in a nucleic acid sequence encoding a polypeptide can be increased, as described in detail in, for example, U.S. Patent Application Publication 2009/0075378 A1 and International Patent Application Publication WO 2008/103475. This approach can be applied to the entire coding region of the nucleic acid sequence, thereby rendering the entire nucleic acid sequence more susceptible to SHM. This approach can be used if relatively little is known about structure activity relationships of an antigen-binding agent (e.g., an antibody).

In another embodiment, the nucleic acid sequence can be modified in order to modulate splicing of the nucleic acid sequence during transcription. In this respect, it may be desirable to express alternate forms of the same polypeptide in a single cell (e.g., secreted and membrane-associated immunoglobulin polypeptides). For example, a recombinant nucleic acid sequence can be generated to include splice donor and/or acceptor sites, which are made available to the splicing machinery of a cell under specific conditions. Such methods are disclosed in, for example, International Patent Application Publication WO 2011/115996.

The nucleic acid sequence encoding a polyepeptide also can be selectively and/or systematically modified through the targeted replacement of regions of interest with synthetic variable regions as described in, for example, U.S. Patent Application Publication 2009/0075378 A1 and International Patent Application Publication WO 2008/103475, which provides for a high density of hot spots and seeds maximal diversity through SHM at specific loci. In another embodiment, a nucleic acid sequence encoding a polypeptide, particularly an immunoglobulin heavy or light chain polypeptide, can be subjected to “chain shuffling.” Chain shuffling (also referred to in the art as “guided selection” or generating “combinatorial antibody libraries”) involves identifying an antibody that binds to an antigen of interest from a library. The nucleic acid sequence encoding a component of an antibody (e.g., the light chain variable region) is then diversified by random- or site-specific-mutation, while the nucleic acid sequence encoding another component of an antibody (e.g., the heavy chain variable region) is fixed (see, e.g., Kang et al., Proc. Natl. Acad. Sci. USA, 88: 11120-11123 (1991)). Chain shuffling on the surface of phage also has been used as a means for humanizing an antibody that binds to an antigen of interest (see, e.g., Jespers et al., Nat. Biotech., 12: 899 903 (1994), U.S. Pat. Nos. 5,565,332 and 6,258,562, U.S. Patent Application Publication 2006/0029594 A1, and International Patent Application Publication WO 93/06213). Methods of using chain shuffling in combination with somatic hypermutation as a means to affinity mature immunoglobulin-encoding nucleic acid sequences are disclosed in, e.g., International Patent Application Publication WO 2011/056864. Any or all of the aforementioned approaches to modify a nucleic acid sequence can be undertaken in conjunction with the inventive method.

In certain embodiments, the nucleic acid sequence encoding a polypeptide is coupled in frame to a nucleic acid sequence encoding a suitable transmembrane domain in order to provide cell-surface display of the polypeptide (e.g., an antibody). For example, for expression in eukaryotic cells, a MHC type 1 transmembrane domain, such as that from H2kk (including peri-transmembrane domain, transmembrane domain, and cytoplasmic domain; NCBI Gene Accession number AK153419), can be coupled in frame to the nucleic acid sequence using standard molecular biology techniques. Likewise, the surface expression of proteins in prokaryotic cells (such as in E. coli and Staphylococcus), insect cells, and yeast is well established in the art (see, e.g., Winter et al., Annu. Rev. Immunol., 12: 433-55 (1994), Pluckthun, A., Bio/Technology, 9: 545-551 (1991), Gunneriusson et al., J. Bacteriol., 78: 1341-1346 (1996), Ghiasi et al., Virology, 185: 187-194 (1991), Boder and Wittrup, Nat. Biotechnol., 15: 553-557 (1997), and Mazor et al., Nat. Biotech., 25(5): 563-565 (2007)).

In other embodiments, a surface displayed polypeptide can be created through the secretion and then binding (or association) of secreted components on the cell surface. Conjugation of the polypeptide to the cell membrane can occur either during protein synthesis or after one or more components of the polypeptide have been secreted from the cell. Conjugation can occur via covalent linkage, by binding interactions (e.g., mediated by specific binding members), or a combination of covalent and non-covalent linkage.

The invention comprises contacting a cell in vitro with the nucleic acid sequence that encodes the polypeptide. An “in vitro” method is conducted using components of an organism that have been isolated from its usual biological context. Examples of in vitro methodology include cell culture, analysis of cellular or subcellular extracts (e.g. wheat germ or reticulocyte extracts), and purification of nucleic and amino acid sequences. In contrast, the term “in vivo” refers to a method that is conducted with living organisms in their normal, intact state, while “ex vivo” refers to methods conducted within or on cells or tissue in an artificial environment outside the organism with minimum alteration of natural conditions.

The cell expresses or can be induced to express Activation Induced Cytidine Deaminase (AID). The term “AID,” as used herein, refers to any protein that is a member of the AID/APOBEC family of RNA/DNA editing cytidine deaminases that are capable of mediating the deamination of cytosine to uracil within a DNA sequence (see, e.g., Conticello et al., Mol. Biol. Evol., 22: 367-377 (2005), and U.S. Pat. No. 6,815,194). In certain embodiments of the invention, AID can be endogenous to the cell. Alternatively, a nucleic acid encoding AID may be provided to a cell which does, or does not, contain an endogenous AID protein. The exogenously provided AID can be a wild-type AID, which refers to a naturally occurring amino acid sequence of an AID protein. Suitable wild-type AID proteins include all vertebrate forms of AID, including, for example, primate, rodent, avian, and bony fish. Representative examples of wild-type AID amino acid sequences include without limitation, human AID (SEQ ID NO: 1 or SEQ ID NO: 2), canine AID (SEQ ID NO: 3), murine AID (SEQ ID NO: 4), rat AID (SEQ ID NO: 5), bovine AID (SEQ ID NO: 6), chicken AID (SEQ ID NO: 7), porcine AID (SEQ ID NO: 8), chimp AID (SEQ ID NO: 9), macaque AID (SEQ ID NO: 10), horse AID (SEQ ID NO: 11), Xenopus AID (SEQ ID NO: 12), pufferfish (fugu) AID (SEQ ID NO: 13), and zebrafish (SEQ ID NO: 14). The use of AID in cells is described in detail in, for example, U.S. Patent Application Publication 2009/0075378 A1 and International Patent Application Publications WO 2008/103474 and WO 2008/103475.

In other embodiments, the exogenously provided AID can be a an “AID mutant” or a “mutant of AID.” As used herein, an “AID mutant” or a “mutant of AID” refers to an AID amino acid sequence that differs from a wild-type AID amino acid sequence by at least one amino acid. A wild-type amino acid sequence can be mutated to produce an AID mutant by any suitable method known in the art, such as, for example, by insertion, deletion, and/or substitution. For example, mutations may be introduced into a nucleic acid sequence encoding wild-type AID randomly or in a site-specific manner. Random mutations may be generated, for example, by error-prone PCR of an AID template sequence. A preferred means for introducing random mutations is the Genemorph II Random Mutagenesis Kit (Stratagene, LaJolla, Calif.). Site-specific mutations can be introduced, for example, by ligating into an expression vector a synthesized oligonucleotide comprising the modified site. Alternately, oligonucleotide-directed site-specific mutagenesis procedures can be used, such as those disclosed in Walder et al., Gene, 42: 133-139 (1986); Bauer et al., Gene, 37: 73-81 (1985); Craik, Biotechniques, 3: 12-19 (January 1985); and U.S. Pat. Nos. 4,518,584 and 4,737,462. A preferred means for introducing site-specific mutations is the QuikChange Site-Directed Mutagenesis Kit (Stratagene, LaJolla, Calif.).

Preferably, an AID mutant is a “functional mutant of AID” or a “functional AID mutant,” which refers to a mutant AID protein which retains all or part of the biological activity of a wild-type AID, or which exhibits increased biological activity as compared to a wild-type AID protein. The biological activity of a wild-type AID includes, but is not limited to, the deamination of cytosine to uracil within a DNA sequence, papillation in a bacterial mutagenesis assay, somatic hypermutation of a target gene, and immunoglobulin class switching. A mutant AID protein can retain any part of the biological activity of a wild-type AID protein. Desirably, the mutant AID protein retains at least 75% (e.g., 75% or more, 80% or more, or 90% or more) of the biological activity of wild-type AID. Preferably, the mutant AID protein retains at least 90% (e.g., 90% or more, 95% or more, or 100% or more) of the biological activity of wild-type AID.

In a preferred embodiment, the mutant AID protein exhibits increased biological activity as compared to a wild-type AID protein. In this respect, the functional AID mutant can display at least a 10-fold improvement in activity as compared to a wild-type AID protein as measured, for example, by a bacterial papillation assay (see, e.g., Nghiem et al., Proc. Natl. Acad. Sci. USA, 85: 2709-2713 (1988), and Ruiz et al., J. Bacteriol., 175: 4985-4989 (1993)). Suitable mutant AID proteins which exhibit increased biological activity as compared to a wild-type AID protein are described in Wang et al., Nat. Struct. Mol. Biol., 16(7): 769-76 (2009), and International Patent Application Publication No. WO 2010/113039.

In other embodiments, the cell can express or be induced to express an AID homolog. The term “AID homolog” refers to the enzymes of the Apobec family and include, for example, Apobec-1, Apobec3C, and Apobec3G (described, for example, in Jarmuz et al., Genomics, 79: 285-296 (2002)).

When the cell is contacted in vitro with a nucleic acid sequence that encodes a polypeptide, expression of AID in the cell will induce somatic hypermutation (SHM) of the nucleic acid sequence. As used herein, “somatic hypermutation” or “SHM” refers to the mutation of a polynucleotide sequence which can be initiated by, or associated with, the action of activation-induced cytidine deaminase (AID), which includes members of the AID/APOBEC family of RNA/DNA editing cytidine deaminases, as described above. SHM can also be initiated by, or associated with, for example, the action of uracil glycosylase and/or error prone polymerases on a polynucleotide sequence of interest. SHM is intended to include mutagenesis that occurs as a consequence of the error prone repair of an initial DNA lesion, including mutagenesis mediated by the mismatch repair machinery and related enzymes. Systems and methods for inducing somatic hypermutation, including nucleic acid and amino acid sequences encoding AID, are described in, e.g., International Patent Application Publication Nos. WO 2008/103475, WO 2008/103474, WO 2003/095636, and WO 2010/113039.

Upon expression of AID in the cell, one or more insertion mutations and/or one or more deletion mutations are introduced in the nucleic acid sequence encoding the polypeptide. The term “insertion mutation” (or “insertion”), as used herein, refers to the addition of one or more nucleotides into a nucleic acid sequence. The term “deletion mutation,” as used herein, refers to the removal or loss of one or more nucleotides from a nucleic acid sequence, and is also referred to in the art as a “gene deletion,” a “deficiency,” or a “deletion.” Both one or more insertion mutations and one or more deletion mutations can be introduced into the nucleic acid sequence as a result of AID expression in the cell. Alternatively, either one or more insertion mutations or one or more deletion mutations can be introduced into the nucleic acid sequence as a result of AID expression in the cell.

In one embodiment, the insertion mutation comprises a duplication of a portion of the nucleic acid sequence encoding the polypeptide. In this regard, any portion of the nucleic acid sequence encoding the polypeptide can be duplicated upon expression of AID in the cell. For example, when the nucleic acid sequence encodes an antibody, or fragment thereof, a portion of the nucleic acid sequence encoding a CDR (e.g., CDR1, CDR2, and/or CDR3) can be duplicated upon expression of AID in the cell. In another embodiment, the duplicated sequence can undergo further mutation events, such as one or more point mutations, one or more deletion mutations, or a combination of one or more point mutations or one or more deletion mutations.

The insertion mutation can comprise the insertion of any suitable number of nucleotides into the nucleic acid sequence. Desirably, the insertion mutation comprises insertion of at least 1 nucleotide (e.g., 2 or more, 5 or more, 10 or more, 15 or more, 20 or more, 25 or more, 30 or more, 40 or more, 45 or more, or 50 or more nucleotides), but no more than 100 nucleotides (e.g., 99 or less, 95 or less, 90 or less, 85 or less, 80 or less, 75 or less, 70 or less, 65 or less, 60 or less, or 55 or less nucleotides). Preferably, the insertion mutation comprises 1-10 nucleotides (e.g., 1, 2, 3, 4, 5, 6, 7, 8, 9, or 10 nucleotides, or a range defined by any two of the foregoing values), 8-15 nucleotides (e.g., 8, 9, 10, 11, 12, 13, 14, or 15 nucleotides, or a range defined by any two of the foregoing values), 15-25 nucleotides (e.g., 15, 16, 17, 18, 19, 20, 21, 22, 23, 24, or 25 nucleotides, or a range defined by any two of the foregoing values), 25-50 nucleotides (e.g., 25, 30, 35, 40, 45, or 50 nucleotides, or a range defined by any two of the foregoing values), 50-80 nucleotides (e.g., 50, 55, 60, 65, 70, 75, or 80 nucleotides, or a range defined by any two of the foregoing values), or 80-100 nucleotides (e.g., 80, 85, 90, 95, 99, or 100 nucleotides, or a range defined by any two of the foregoing values). More preferably, the insertion mutation comprises an insertion of about 3-24 nucleotides (e.g., 3, 4, 5, 6, 7, 8, 9, 12, 15, 18, 21, or 24 nucleotides) into the nucleic acid sequence. Most preferably, the insertion mutation comprises an insertion of about 3-9 nucleotides (e.g., 3, 4, 5, 6, 7, 8, or 9 nucleotides) into the nucleic acid sequence. In a particularly preferred embodiment, the size of the insertion mutation occurs in multiples of three nucleotides (or “triplets”), so as to preserve the reading frame of the polypeptide-coding region.

Similarly, the deletion mutation can comprise the deletion of any suitable number of nucleotides from the nucleic acid sequence. Desirably, the deletion mutation comprises deletion of at least 1 nucleotide (e.g., 2 or more, 5 or more, 10 or more, 15 or more, 20 or more, 25 or more, 30 or more, 40 or more, 45 or more, or 50 or more nucleotides), but no more than 100 nucleotides (e.g., 99 or less, 95 or less, 90 or less, 85 or less, 80 or less, 75 or less, 70 or less, 65 or less, 60 or less, or 55 or less nucleotides). Preferably, the deletion mutation comprises 1-10 nucleotides (e.g., 1, 2, 3, 4, 5, 6, 7, 8, 9, or 10 nucleotides, or a range defined by any two of the foregoing values), 8-15 nucleotides (e.g., 8, 9, 10, 11, 12, 13, 14, or 15 nucleotides, or a range defined by any two of the foregoing values), 15-25 nucleotides (e.g., 15, 16, 17, 18, 19, 20, 21, 22, 23, 24, or 25 nucleotides, or a range defined by any two of the foregoing values), 25-50 nucleotides (e.g., 25, 30, 35, 40, 45, or 50 nucleotides, or a range defined by any two of the foregoing values), 50-80 nucleotides (e.g., 50, 55, 60, 65, 70, 75, or 80 nucleotides, or a range defined by any two of the foregoing values), or 80-100 nucleotides (e.g., 80, 85, 90, 95, 99, or 100 nucleotides, or a range defined by any two of the foregoing values). More preferably, the deletion mutation comprises a deletion of 3-24 nucleotides (e.g., 3, 4, 5, 6, 7, 8, 9, 12, 15, 18, 21, or 24 nucleotides) from the nucleic acid sequence. Most preferably, the deletion mutation comprises a deletion of 3-9 nucleotides (e.g., 3, 4, 5, 6, 7, 8, or 9 nucleotides) from the nucleic acid sequence. In a particularly preferred embodiment, the size of the deletion mutation occurs in multiples of three nucleotides (or “triplets”), so as to preserve the reading frame of the polypeptide-coding region.

As discussed herein, the nucleic acid sequence preferably encodes an immunoglobulin heavy chain polypeptide or an immunoglobulin light chain polypeptide. In this regard, the one or more insertion mutations and/or the one or more deletion mutations can be introduced at any suitable location of the nucleic acid sequence encoding the immunoglobulin heavy or light chain polypeptide. For example, the one or more insertion mutations and/or the one or more deletion mutations can be introduced into the region of the nucleic acid sequence that encodes the variable region or the constant region of the immunoglobulin heavy or light chain. Preferably, the one or more insertion mutations and/or the one or more deletion mutations are introduced into the region of the nucleic acid sequence that encodes the variable region of the immunoglobulin heavy or light chain polypeptide. In this respect, the one or more insertion mutations and/or the one or more deletion mutations desirably occur in one or more complementarity determining regions (CDRs) of the immunoglobulin heavy or light chain polypeptide (e.g., CDR1, CDR2, or CDR3). The one or more insertion mutations and/or the one or more deletion mutations can occur in any one, two, or all three CDRs in any combination. For example, an insertion mutation can occur in CDR1 in combination with a deletion mutation in CDR2 or CDR3. Alternatively, a deletion mutation can occur in each of CDR1, CDR2, and CDR3. In another embodiment, an insertion mutation can occur in each of CDR1, CDR2, and CDR3. Furthermore, an insertion mutation can occur in CDR3, or a deletion mutation can occur in CDR3. In a preferred embodiment, the one or more insertion mutations and/or the one or more deletion mutations occur in CDR3 of the immunoglobulin heavy or light chain polypeptide. In a particularly preferred embodiment, the one or more insertion mutations result in the insertion of one to eight amino acid residues into the immunoglobulin heavy or light chain polypeptide. In addition or alternatively, the one or more deletion mutations preferably result in the deletion of one to eight amino acid residues from the immunoglobulin heavy or light chain polypeptide.

The introduction of one or more insertion mutations and/or one more deletion mutations in a nucleic acid sequence desirably produces a change in at least one property of the polypeptide encoded thereby. The property can contribute to, for example, the biological function of the polypeptide (e.g., enzymatic activity, ligand binding, antigen-binding, or DNA-binding), polypeptide folding, and/or stability of the polypeptide in vivo. In this respect, the polypeptide can be screened for a desired property (e.g., a selectable or improved phenotype) using a variety of standard physiological, pharmacological, and/or biochemical procedures. Such assays include for example, biochemical assays such as binding assays, fluorescence polarization assays, solubility assays, folding assays, thermostability assays, proteolytic stability assays, and enzyme activity assays (see, e.g., Glickman et al., J. Biomolecular Screening, 7(1): 3-10 (2002), and Salazar et al., Methods. Mol. Biol., 230: 85-97 (2003)), and a range of cell based assays including signal transduction, motility, whole cell binding, flow cytometry, and fluorescent activated cell sorting (FACS) based assays. When the nucleic acid sequence encodes an immunoglobulin heavy or light chain polypeptide, or fragment thereof, the phenotype/function of the immunoglobulin or fragment thereof can be analyzed using any suitable technique, such as, for example, enzyme-linked immunosorbant assays (ELISA), enzyme-linked immunosorbant spot (ELISPOT) assay, gel detection and fluorescent detection of mutated IgH chains, Scatchard analysis, Biacore analysis, western blots, polyacrylamide gel (PAGE) analysis, radioimmunoassays, and the like, which can determine binding affinity, binding avidity, and other properties.

In accordance with the invention, the nucleic acid sequence can be provided to a cell in the form of a vector, such as a plasmid, episome, cosmid, viral vector (e.g., retroviral or adenoviral), or phage. Suitable vectors and methods of vector preparation are well known in the art (see, e.g., Sambrook and Russell, Molecular Cloning: A Laboratory Manual, Cold Spring Harbor Laboratory Press, 3^{rd} Edition (2001), and Maniatis et al., Cell Biology: A Comprehensive Treatise, Vol. 3, Gene Sequence Expression, Academic Press, New York, pp. 563-608 (1980)). Preferably, the vector is a replicable genetic display package. By “replicable genetic display package” is meant a biological particle comprising genetic information which provides the particle with the ability to replicate. The particle can display on its surface at least part of the polypeptide encoded by the nucleic acid sequence described above. In certain embodiments, the replicable genetic display package is a viral vector or a bacteriophage.

In addition to the nucleic acid encoding a polypeptide, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include the T7 bacterial expression system, pBAD (araA) bacterial expression system, the cytomegalovirus (CMV) promoter, the SV40 promoter, and the RSV promoter. Inducible promoters include, for example, the Tet system (see, e.g., U.S. Pat. Nos. 5,464,758 and 5,814,618), the Ecdysone inducible system (see, e.g., No et al., Proc. Natl. Acad. Sci., 93: 3346-3351 (1996)), the T-REx™ system (Life Technologies, Carlsbad, Calif.), the LacSwitch® System (Stratagene, San Diego, Calif.), and the Cre-ERT tamoxifen inducible recombinase system (see, e.g., Indra et al., Nuc. Acid. Res., 27: 4324-4327 (1999), Fuhrmann-Benzakein et al., Nuc. Acid. Res., 28(23): e99 (2000), Kramer & Fussenegger, Methods Mol. Biol., 308: 123-144 (2005), and U.S. Pat. No. 7,112,715.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see, e.g., Fundamental Immunology, 3rd Edition, W. E. Paul (ed.), Raven Press, New York (1993), pages 353-363, and U.S. Pat. No. 5,885,827).

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150:1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359

In certain embodiments, the nucleic acid encoding the polypeptide is provided to a cell in the form of an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Life Technologies (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cells chromosome. Such integrating expression vectors may utilize the endogenous expression control sequences of the host cell's chromosomes to effect expression of the desired protein. Examples of vectors that integrate in a site-specific manner include, for example, components of the flp-in system from Life Technologies (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Life Technologies (Carlsbad, Calif.), and pCI or pFN10A (ACT) FLEXI™ from Promega (Madison, Wis.).

Viral vectors also can be used in the inventive method. Representative commercially available viral expression vectors include, but are not limited to, the adenovirus-based Per.C6 system available from Crucell, Inc. (Leiden, The Netherlands), the lentiviral-based pLP1 from Life Technologies (Carlsbad, Calif.), and the retroviral vectors pFB-ERV plus pCFB-EGSH from Stratagene (La Jolla, Calif.).

The vector comprising the nucleic acid encoding the polypeptide can be introduced into any host cell that is capable of expressing the polypeptide, including any suitable prokaryotic or eukaryotic cell. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the vector comprising the nucleic acid sequence encoding the polypeptide is introduced into a eukaryotic cell. Suitable eukaryotic cells are known in the art and include, for example, yeast cells, insect cells, and mammalian cells. Examples of suitable yeast cells include those from the genera Hansenula, Kluyveromyces, Pichia, Rhino-sporidium, Saccharomyces, and Schizosaccharomyces. Preferred yeast cells include, for example, Saccharomyces cerivisae and Pichia pastoris. Suitable insect cells are described in, for example, Kitts et al., Biotechniques, 14: 810-817 (1993), Lucklow, Curr. Opin. Biotechnol., 4: 564-572 (1993), and Lucklow et al., J. Virol., 67: 4566-4579 (1993). Preferred insect cells include Sf-9 and HIS (Life Technologies, Carlsbad, Calif.).

Preferably, mammalian cells are utilized in the inventive method. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa cells, mouse L-929 cells, and BHK or HaK hamster cell lines, all of which are available from the ATCC. Preferably, the cell is an HEK-293 cell. The selection of suitable mammalian host cells and methods for transformation, culture, amplification, screening, and purification of cells are known in the art.

In a preferred embodiment, the mammalian cell is a human cell. For example, the mammalian cell can be a human lymphoid or lymphoid derived cell line, such as a B-cell or a cell line of pre-B lymphocyte origin. Examples of human lymphoid cells lines include RAMOS(CRL-1596), Daudi (CCL-213), EB-3 (CCL-85), DT40 (CRL-2111), 18-81 (Jack et al., Proc. Natl. Acad. Sci. USA, 85: 1581-1585 (1988)), and Raji cells (CCL-86), and derivatives thereof.

The nucleic acid sequence encoding a polypeptide may be introduced into a cell by “transfection,” “transformation,” or “transduction.” The terms “transfection,” “transformation,” or “transduction,” as used herein, refer to the introduction of one or more exogenous polynucleotides into a host cell by physical or chemical methods. Many transfection techniques are known in the art and include, for example, calcium phosphate DNA co-precipitation (see, e.g., Methods in Molecular Biology, Vol. 7, E. J. Murray (ed.), Gene Transfer and Expression Protocols, Humana Press (1991)), DEAE-dextran, electroporation, cationic liposome-mediated transfection, tungsten particle-facilitated microparticle bombardment (see, e.g., Johnston, Nature, 346: 776-777 (1990)), and strontium phosphate DNA co-precipitation (see, e.g., Brash et al., Mol. Cell. Biol., 7: 2031-2034 (1987)).

The invention provides a method of producing an immunoglobulin. The method comprises (a) providing a cell in vitro that expresses or can be induced to express AID, (b) contacting the cell in vitro with the aforementioned nucleic acid sequence encoding an immunoglobulin heavy chain polypeptide and the aforementioned nucleic acid sequence encoding an immunoglobulin light chain polypeptide, and (c) expressing AID in the cell, whereupon an immunoglobulin comprising a heavy chain polypeptide and a light chain polypeptide is produced in the cell. Descriptions of the cell, AID, and nucleic acid sequences set forth above in connection with other embodiments of the invention also are applicable to those same aspects of the aforesaid method. The invention also provides an isolated or purified nucleic acid sequence which encodes an immunoglobulin light chain polypeptide or an immunoglobulin heavy chain polypeptide prepared in accordance with the inventive methods described herein.

The following examples further illustrate the invention but, of course, should not be construed as in any way limiting its scope.

### Example 1

This example describes the generation of insertion and deletion mutations in a nucleic acid sequence encoding a heavy chain of a human nerve growth factor (NGF) antibody in accordance with the inventive method.

Plasmids containing (a) a nucleic acid sequence encoding an anti-NGF heavy chain polypeptide (HC) (SEQ ID NO: 16) and (b) a nucleic acid sequence encoding a corresponding light chain polypeptide (LC) were co-expressed with a plasmid containing a nucleic acid sequence encoding an AID protein (SEQ ID NO: 15) in an HEK-293 c18 cell line. The HC and LC sequences were affinity matured in iterative rounds of FACS selection in the presence of diminishing concentrations of fluorescently labeled antigen.

In this respect, the starting HEK-293 c18 cell line expressing the HC and LC together with AID was generated by seeding a T75 culture flask with 3×10⁶ HEK-293 c18 cells in 10 mL Dulbecco's Modified Eagle's Medium (DMEM) containing 10% fetal bovine serum (FBS) (Life Technologies, Carlsbad, Calif.). Plasmid constructs were transfected using 500 μL OptiMEM (Life Technologies, Carlsbad, Calif.) and 20 μL HD-Fugene (Roche Diagnostics Corporation, Indianapolis, Ind.). Three days post-transfection, cell growth medium was exchanged with 10 mL DMEM medium containing 10% FBS, 50 μg/mL GENETICIN™ aminoglycoside antibiotic (Life Technologies, Carlsbad, Calif.), 10 μL/mL Antibiotic-Antimycotic Solution (Sigma-Aldrich, St. Louis, Mo.), 1.5 μg/mL puromycin, 15 μg/mL blasticidin, and/or 350 μg/mL hygromycin, and the cells were incubated for approximately four weeks with periodic reseeding and exchange of the cell culture medium.

A HEK-293 c18 cell line expressing a surface bound anti-NGF antibody was incubated with 50 nM of biotinylated antigen (Ag-bio) recomplexed with 200 nM of tetrameric NGF-fluorescein isothiocyanate (FITC) for 10 minutes at room temperature in 1 mL phosphate buffered saline (PBS) with 1% bovine serum albumin (BSA). 1×10⁸ cells were spun down at 1100 rpm and resuspended in 1 mL of the above-described complex and incubated for 30 minutes at 4° C. Surface IgG was detected by adding a 1:500 dilution of goat α-human IgG-PE (γ chain-specific) and 1:100 of marina blue-labeled streptavidin (SA-MB) (Life Technologies, Carlsbad, Calif.) was added to gate out library cells with non-specific streptavidin (SA) binding. Cells were incubated for 30 minutes at 4° C., spun down at 1100 rpm and resuspended in 1 mL of 2 μg/mL 4′,6-diamidino-2-phenylindole fluorescent stain (DAPI) in PBS/1% BSA. FACS selection of FITC-positive cells was performed as described herein. During Rounds 2-12 of FACS selection, 2×10⁷ cells were incubated with FITC-labeled antigen for 30 minutes at 4° C. at concentrations determined empirically from FACS binding analysis, and ranging from 100 nM NGF-FITC (Round 2) to 30 μM NGF-FITC (Round 12).

The distribution of insertion and deletion mutations observed during affinity maturation of SEQ ID NO: 16 was examined by Sanger sequencing. Approximately 50 HC nucleic acid sequences were obtained following each round of FACS selection. DNA templates were isolated from 3.3×10⁴ cells by PCR amplification using Accuprime PFX polymerase for 25 to 30 cycles. Oligonucleotide primers were used that encompass from approximately 140 nucleotides 5′ to the ATG start codon through 30 nucleotides 3′ to the junction of the variable/constant regions, to yield amplicons of approximately 550 nucleotides in length for both the HC and LC. Variant sequences were analyzed from the start codon through the first 150-155 amino acids of the HC, or through 135 amino acids of the LC (up to the variable/constant region junction in each case).

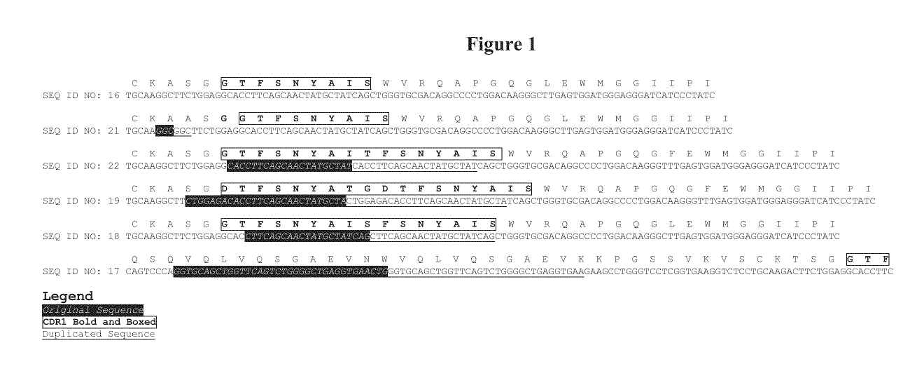

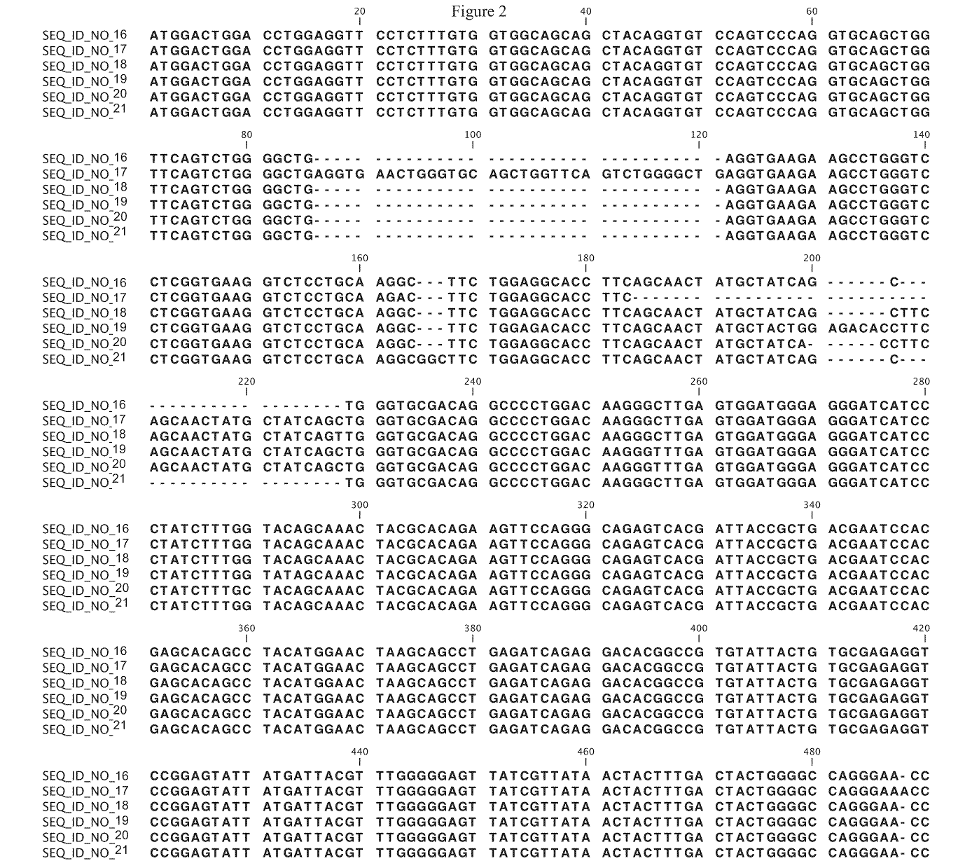

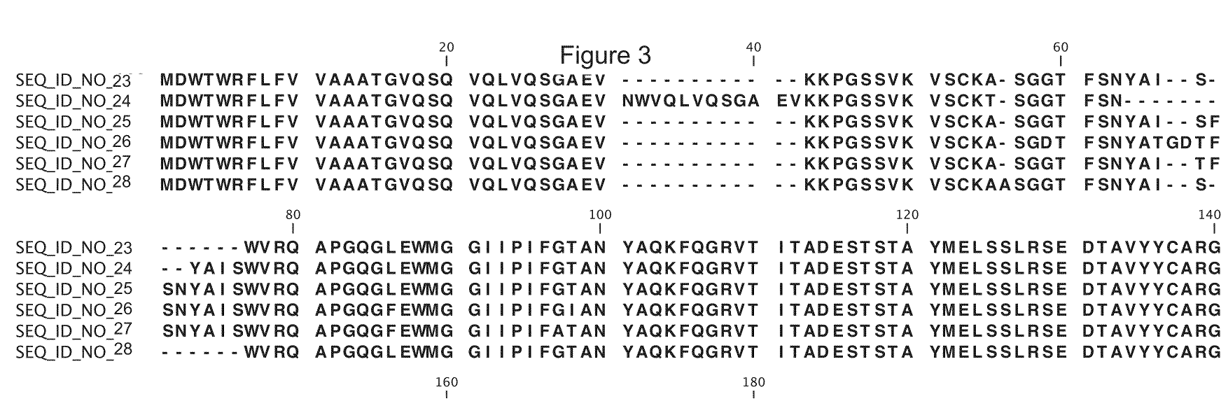

Multiple nucleic acid sequence variants of SEQ ID NO: 16 were isolated from the anti-NGF antibody affinity maturation corridors described above (e.g., SEQ ID NO: 17, SEQ ID NO: 18, SEQ ID NO: 19, SEQ ID NO: 20, SEQ ID NO: 21, and SEQ ID NO: 22), alignments of which are shown in FIGS. 1 and 2. The amino acid sequences encoded by SEQ ID NOs: 16, 17, 18, 19, 20, and 21 are SEQ ID NOs: 23, 24, 25, 26, 27, and 28, respectively, and are shown in FIG. 3.

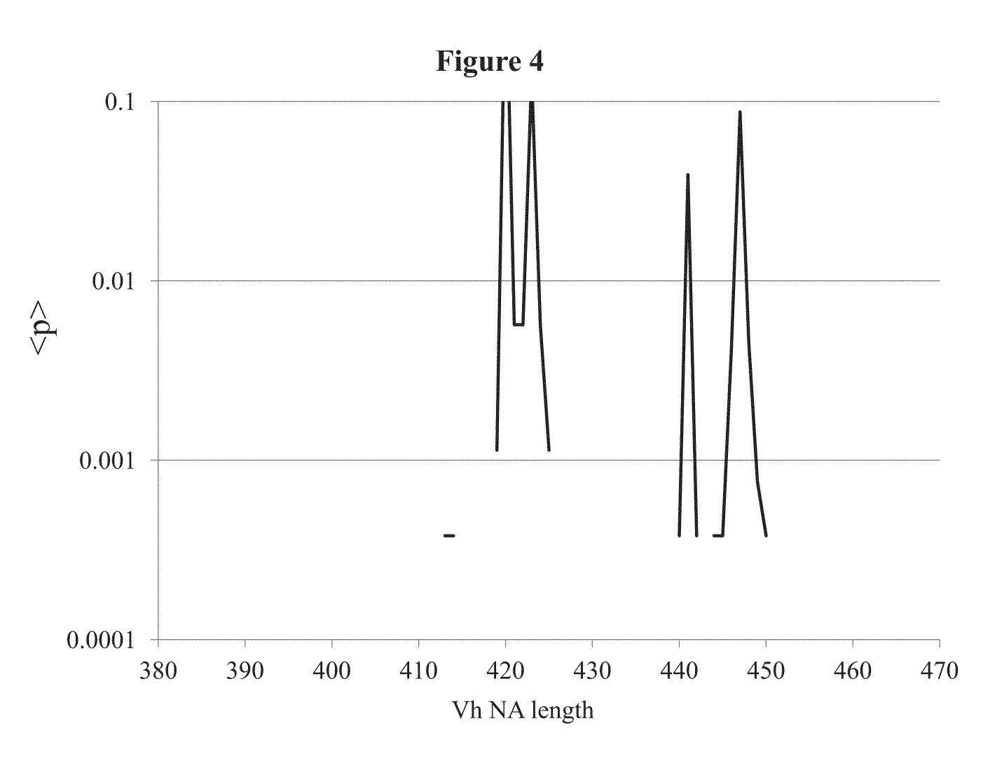

Multiple insertion and deletion mutations were observed in the recovered sequences during rounds 5-9 of affinity maturation, some of which were centered on the junction between framework region 1 (FW1) and CDR1 of the HC. The distribution of heavy chain variable region (Vₕ) lengths from nucleic acid sequences isolated during affinity maturation to NGF is shown in FIG. 4. Of the 4,128 isolated sequences, numerous insertion and deletion mutations were observed, with Vₕ regions varying in length by up to 14 amino acids or 42 nucleotides.

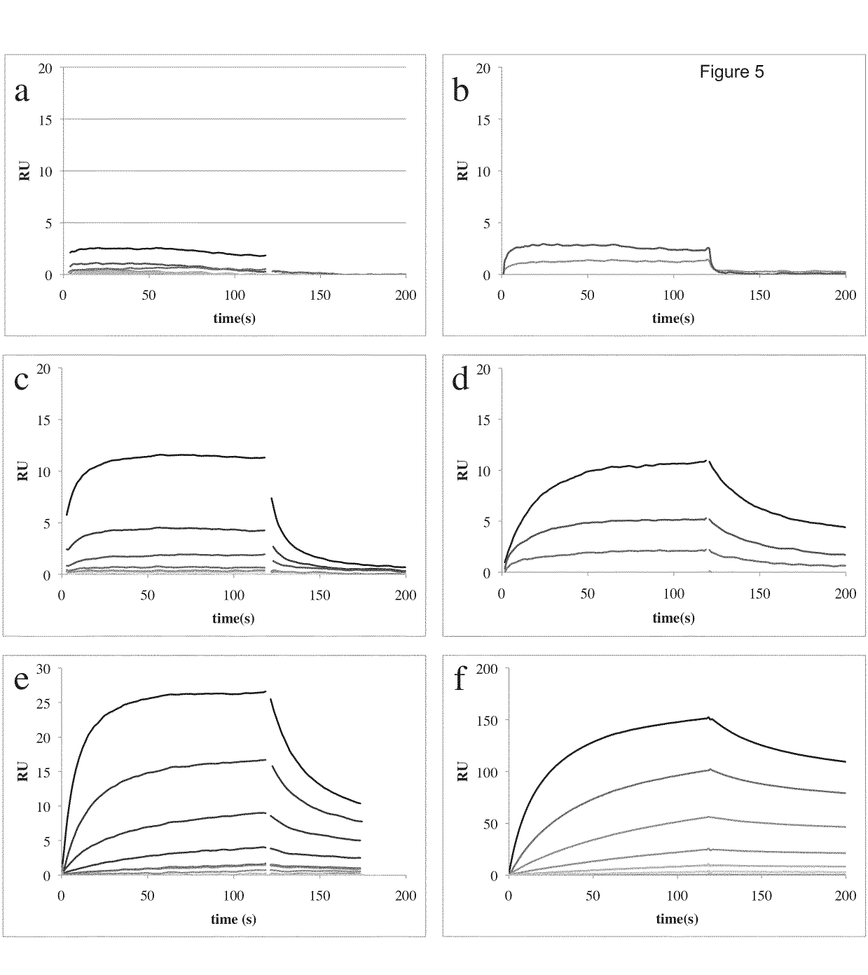

Nucleic acid sequences encoding HC variants containing a CDR1 insertion mutation (i.e., SEQ ID NO: 17, SEQ ID NO: 18, SEQ ID NO: 19, SEQ ID NO: 21, and SEQ ID NO: 22) were expressed and purified, along with the nucleic acid sequence encoding the parental HC (SEQ ID NO: 16), for characterization of kinetic constants using a Biacore T200 assay (GE Healthcare). Each of four flow cells on a Series S CM5 chip was immobilized with approximately 1,000 RU anti-human IgG (Fc). Antibodies (about 1 mg/mL) were captured for 60 seconds at a flow rate of 10 mL/min. βNGF was diluted in running buffer (HBS-EP+ buffer [0.2 M (4-(2-hydroxyethyl)-1-piperazineethanesulfonic acid) (HEPES)/3 M NaCl/60 mM EDTA/1% polysorbate 20, pH 7.6], 0.1% BSA, pH 7.4) starting approximately at a 10-fold higher concentration than each antibody's K_{d}. Each βNGF concentration was passed over all flow cells for 120 seconds at 30 mL/min, then allowed to dissociate for 600 seconds. Surfaces were regenerated with 3 M MgC₁₂ for 180 seconds. Association and dissociation kinetic constants (“kₒₙ” and “k_{off}”) and steady-state affinity (K_{d}) were derived from the resulting sensorgrams using Biacore T200 Evaluation Software version 1.0 (see FIGS. 5a-5f). Heavy chain polypeptides encoded by the nucleic acid sequences containing a CDR1 insertion mutation exhibited about a 20-100 fold improvement in binding affinity relative to the antibody containing the parental HC.

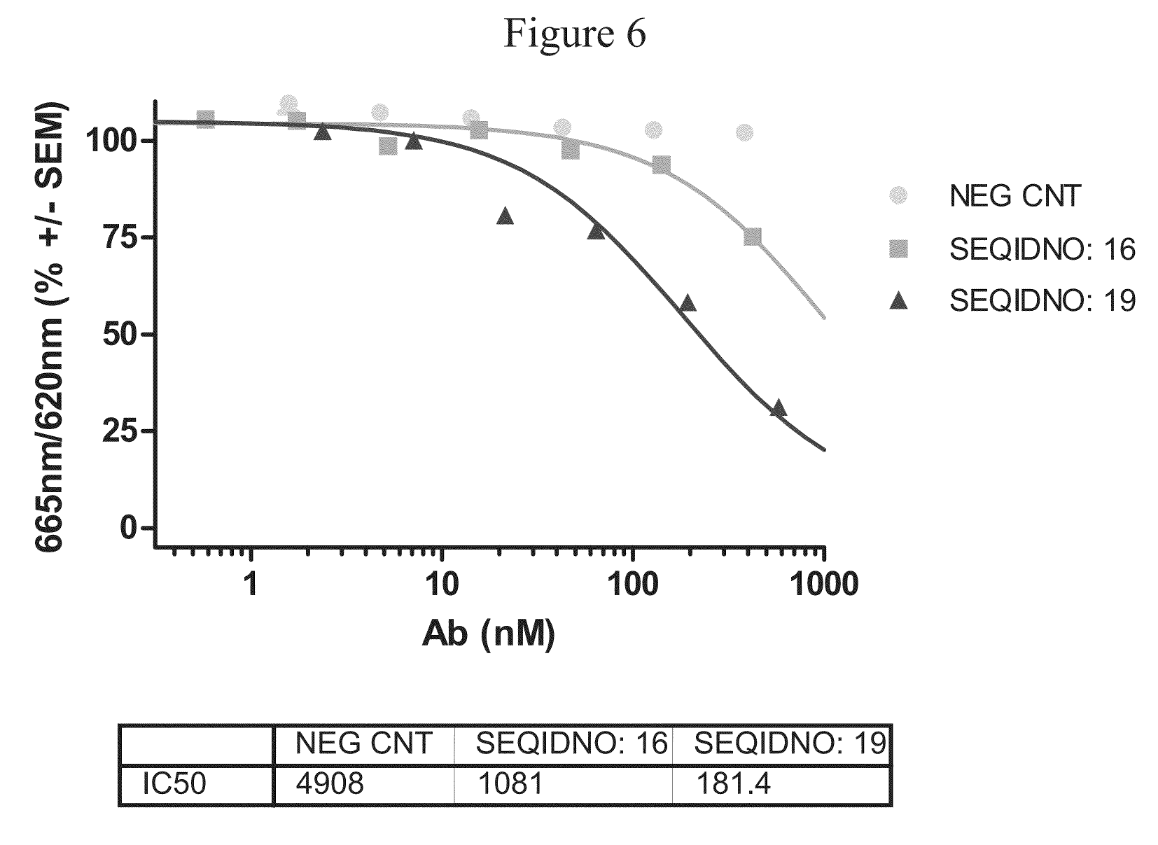

The binding affinity rank order of anti-NGF antibodies with and without CDR1 insertions was determined in a homogeneous time resolved fluorescence (HTRF) assay. A blocking (reference) antibody to βNGF (Tanezumab) was biotinylated, purified, and subsequently labeled with N-hydroxysuccinimide-activated Cryptate using HTRF™ Cryptate Labeling Kit following the manufacturer's protocol (Cisbio Bioassays, Bedford, Mass.). The biotinylated reference antibody was mixed with Streptavidin-XL665 (Cisbio Bioassays, Bedford, Mass.), and the test antibody was added at varying concentrations, followed by incubation with labeled βNGF at room temperature for 2.5 minutes in PBS, pH7.2, 0.1% BSA. The reaction was read in a ProxiPlate-384 Plus (Perkin Elmer, Waltham, Mass.) using an Envision plate reader (Perkin Elmer) (320 nm excitation, dual emission at 620 nm and 665 nm). Binding of βNGF to the reference antibody was represented as the ratio of emission at 665 nm/620 nm. To determine the IC₅₀ values of the test antibodies, the 665 nm/620 nm emission ratios were fitted by a three parameter inhibitory curve using GraphPad Prism (GraphPad Software, La Jolla, Calif.). The results of this assay are shown in FIG. 6, which demonstrates that the HC containing a CDR1 insertion encoded by SEQ ID NO: 19 exhibits an IC₅₀ that is 6-fold more potent that the same HC which lacks the CDR1 mutation encoded by SEQ ID NO: 16.

The results of this example demonstrate that the inventive method can be used to generate insertion mutations in an antibody-encoding nucleic acid sequence that increase the affinity of the antibody.

### Example 2

This example describes the generation of insertion and deletion mutations in a nucleic acid sequence encoding a heavy chain of a human IL-17a antibody in accordance with the inventive method.

A plasmid containing a nucleic acid sequence (SEQ ID NO: 29) encoding an anti-IL17a heavy chain polypeptide (HC) and a plasmid containing a nucleic acid sequence encoding a corresponding light chain polypeptide (LC) were co-expressed with a plasmid containing a nucleic acid sequence encoding an AID protein (SEQ ID NO: 15) in a HEK-293 c18 cell line. The HC and LC sequences were affinity matured in iterative rounds of FACS selection in the presence of diminishing concentrations of fluorescently labeled antigen. Specifically, 30 to 50 million cells were pelleted at 100×g and resuspended in 100 mL PBS/1% BSA plus DyLight 649-labeled WFP-IL17a, and incubated at 4° C. or 25° C. for 1 to 2 hours. Cells were spun down and resuspended in 5 mL PBS/1% BSA plus 1:500 dilution of PE-labeled anti-human IgG (Rockland Immunochemicals, Gilbertsville, Pa.) at 4° C. or 25° C. for 30 minutes. Following a final pelleting, cells were resuspended in 2 mL PBS/1% BSA/0.1% DAPI. Flow cytometry was effected using a BD Influx at a flow rate of ˜10,000 cells/sec, set to capture approximately 0.5% of the cell population. The positively selected cell population was expanded and subjected to iterative rounds of FACS selection. Early in the maturation process, 3 to 10 nM antigen was used. Lower concentrations were subsequently employed later in the process, such that 1 pM antigen was used in the final round of FACS selection.

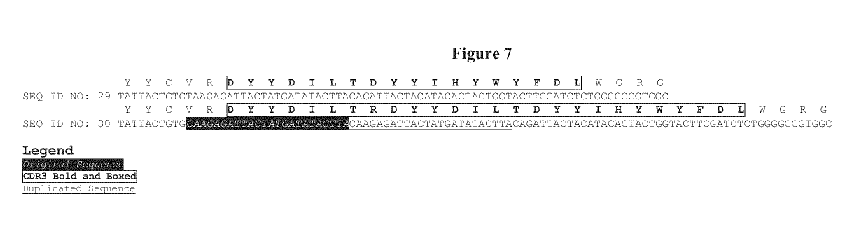

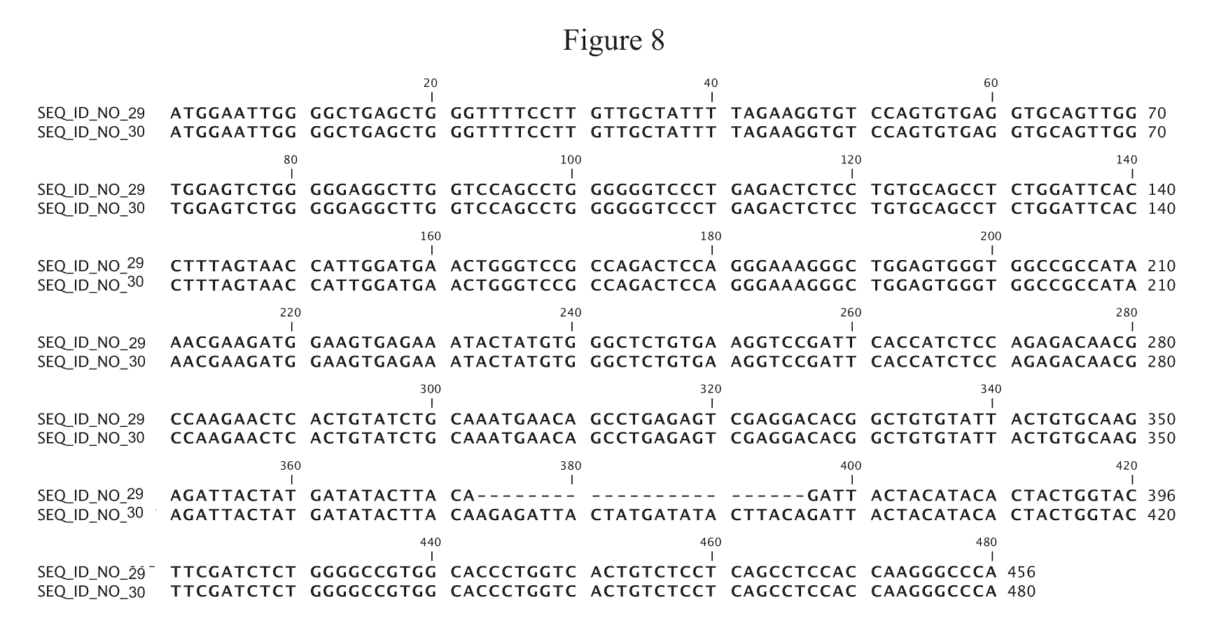

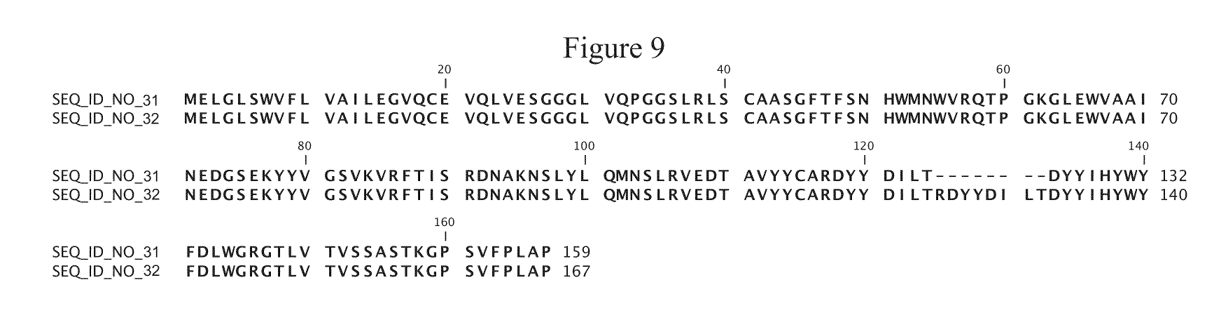

FIG. 7 shows a sequence alignment of a portion of the HC sequences (SEQ ID NO: 29 and SEQ ID NO: 30) recovered from the anti-IL17a affinity maturation corridor described above. Approximately 50 HC sequences were obtained following each round of FACS selection described above, which led to the identification of an enriched HC nucleic acid sequence (SEQ ID NO: 30). FIG. 7 shows that a segment of the HC CDR3 is duplicated, which led to a significant increase in affinity for the antigen (discussed below). FIGS. 8 and 9 show an alignment of the nucleic acid sequences (SEQ ID NO: 29 and SEQ ID NO: 30) and amino acid sequences (SEQ ID NO: 31 and SEQ ID NO: 32) of the entire heavy chain variable region domain, with the insertion visible in the CDR3 of SEQ ID NO: 30 and SEQ ID NO: 32.

The effect of an HC CDR3 insertion on antibody binding affinity to IL-17a was analyzed using a KinExA 3000 assay (Sapidyne Instruments, Boise, Id.), and the results were compared to IL-17a binding of an antibody comprising an HC which lacks the CDR3 insertion (encoded by SEQ ID NO: 29). KinExA technology measures the unbound/free receptor molecule in solution phase. Measuring binding events in the solution phase with micro beads for maximized surface area avoids mass transport limitations and mobility effects inherent to methods which measure binding to a solid phase. For each experiment, 50 μg of human IL-17a was amine-coupled to 50 mg of UltraLink Biosupport beads (Thermo Scientific, Waltham, Mass.; Catalog No. 53110). A constant concentration of antibody (sufficient to produce 0.8 V-1.2 V of signal) was incubated to equilibrium (length of incubation varies for each antibody and is depended on affinity) with titrated antigen in sample buffer (1×PBS, pH 7.4, 0.02% NaN₃, 0.1% BSA). Antibody-antigen solution is then flown over antigen-coupled beads at a rate of 0.25 mL/min. Free antibody captured by beads was detected using Cy5-conjugated AffiniPure Donkey Anti-Human IgG (H+L) (Jackson ImmunoResearch, West Grove, Pa.; Catalog No. 709-175-149). Each of the K_{d} and/or ABC (active binding concentration) of antibody was obtained from non-linear regression analysis using a one-site homogeneous binding model in the KinExA Pro Software.

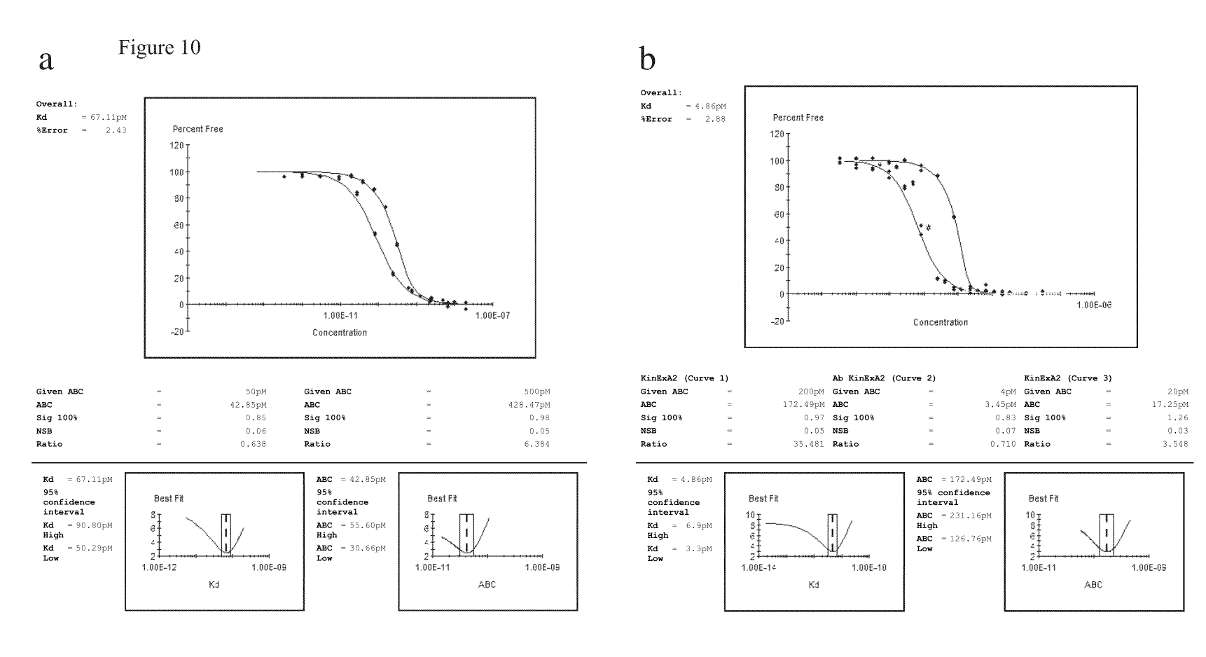

In order to achieve the most accurate measurement for each antibody, each K_{d} controlled curve (where antibody concentration was below the K_{d}) was combined with the receptor controlled curve (where antibody concentration was well above the K_{d}) in N-curve analysis. FIG. 10a shows the N-curve analysis for the antibody containing the HC lacking the CDR3 insertion (encoded by SEQ ID NO: 29), with a measured K_{d}=67 pM. FIG. 10b shows the N-curve analysis for the antibody containing the HC with the CDR3 insertion (encoded by SEQ ID NO: 30), with a measured K_{d}=5 pM, which was approximately a 13-fold improvement in binding affinity relative to the antibody containing the HC encoded by SEQ ID NO: 29.

The distribution of insertion and deletion mutations observed during affinity maturation of SEQ ID NO: 29 was examined by Sanger sequencing. Approximately 50 HC sequences were obtained following each round of FACS selection. DNA templates were isolated from 3.3×10⁴ cells by PCR amplification using Accuprime PFX polymerase for 25 to 30 cycles. Oligonucleotide primers were used that encompass from approximately 140 nucleotides 5′ to the ATG start codon through 30 nucleotides 3′ to the junction of the variable/constant regions, to yield HC and LC amplicons of approximately 550 nucleotides in length. Affinity matured sequences were analyzed from the start codon through the first 150-155 amino acids of the HC, or through 135 amino acids of the LC (up to the variable/constant region junction in each case). The background frequency of PCR errors was measured at 5.2×10⁻⁵ based on the number of mutations observed in 200 HC templates rescued from HEK293 cells in the absence of AID.

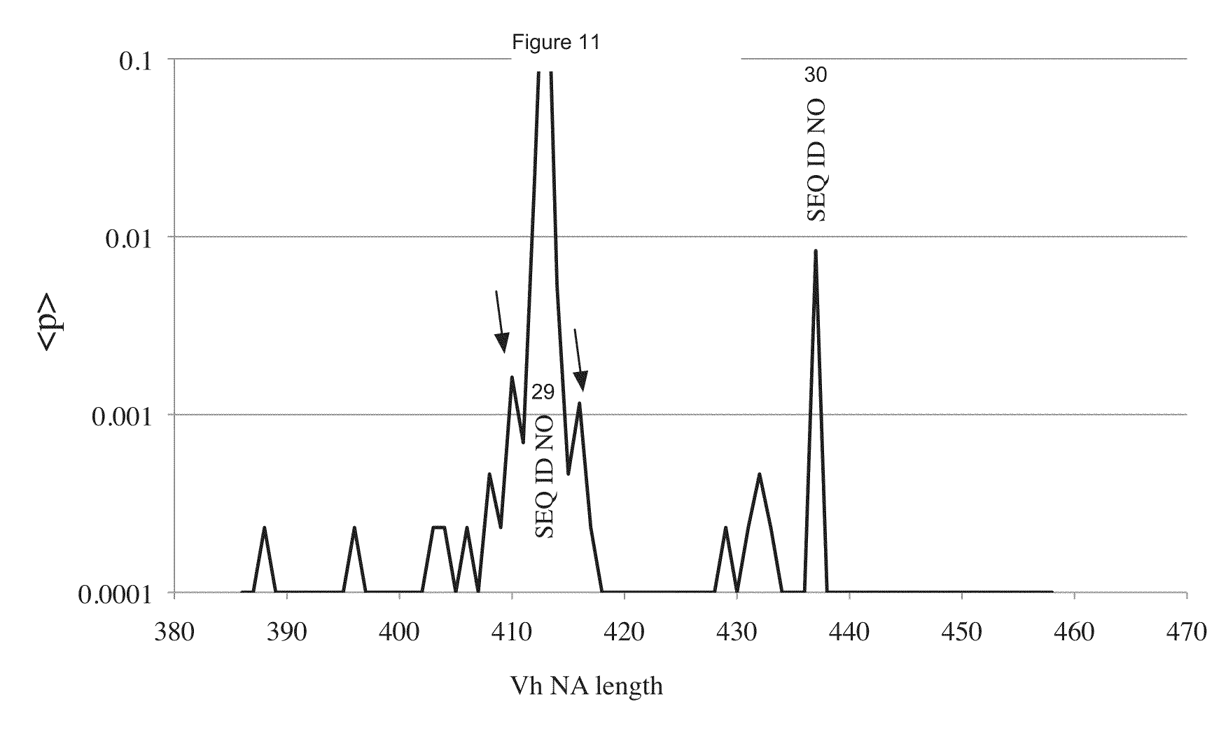

FIG. 11 is a graph of the distribution of Vₕ region lengths from cells isolated during affinity maturation. From the 4,321 isolated sequences, numerous insertion and deletion mutations were observed, with Vh regions varying in length by up to 17 amino acids (51 nucleotides). Single codon insertion and deletion mutations were frequently observed during affinity maturation (highlighted by arrows in FIG. 11), with multiple instances of insertion and/or deletion mutations located in the signal peptide (insertion, SEQ ID NO: 33), CDR1 (deletion, SEQ ID NO: 34), FW1 (insertion, SEQ ID NO: 35), and CDR2 (deletion, SEQ ID NO: 51).

The rank order of binding affinity for the anti-IL17a antibodies described above was determined by a homogeneous time resolved fluorescence (HTRF) assay. In particular, an IL-17 antigen linked to wasabi fluorescent protein (WFP) (see, e.g., Ai et al., BMC Biol., 6:13 (2008)) was labeled with N-hydroxysuccinimide activated cryptate (Eu3+-TBP-NHS Cryptate) using an HTRF™ Cryptate Labeling Kit following the manufacturer's protocol (Cisbio Bioassays, Bedford, Mass.). A biotinylated version of the second reference antibody was linked to Streptavidin-XL665 (Cisbio Bioassays, Bedford, Mass.) and subsequently mixed with each of the aforementioned anti-IL17a antibodies at various concentrations. The antibodies were then incubated with the labeled antigen overnight at room temperature. At the end of the assay, the reaction was read in a ProxiPlate-384 Plus (Perkin Elmer, Waltham, Mass.) using an EnVision Multilabel Plate Reader (PerkinElmer, Waltham, Mass.). The binding of the labeled antigen and the reference antibody was determined as the ratio of absorbance at 665 nm to 620 nm. The ratios were plotted against the concentrations of the tested antibodies, and the IC₅₀ for each tested antibody was determined by inhibitory curve fitting using GraphPad Prism software (GraphPad Software, Inc., La Jolla, Calif.). The results of this assay demonstrate that the antibody containing an HC with the CDR3 insertion (encoded by SEQ ID NO: 30) exhibited an IC₅₀ that was 6-fold more potent than an antibody containing the same HC but which lacks the CDR3 mutation (encoded by SEQ ID NO: 29).

A cytokine release assay using HT1080 cells, NIH 3T3 cells, or primary synovial fibroblast cells from rheumatoid arthritis patients (RA SFB) also was used to demonstrate that immunoglobulin heavy chain (HC) and light chain (LC) polypeptides described above can form antibodies that bind to human IL-17. IL-6 release from NIH3T3 and HT-1080 cells was quantified by ELISA. Cells were seeded in a 96-well assay plate at 1×10⁴ cells/well and were then treated for 24 hours with (i) purified human Myc-IL-17a (APE280, 52 pM for NIH3T3 cells or 200 pM for HT1080 cells), (ii) human recombinant TNFa (R&D Systems, Inc., Minneapolis, Minn., 0.5 ng/mL; NIH3T3 cells only), and (iii) the anti-IL17a antibodies described above at various concentrations (all in 100 ml DMEM/10% FCS). After treatment, 10 mL of supernatant from each well was analyzed by ELISA (eBioscience, Inc., San Diego, Calif.) for mouse IL-6 quantification following the manufacturer's protocol. The IL-6 levels were normalized to the negative control, in which no anti-IL-17a antibody was present during the treatment. The normalized IL-6 levels were plotted versus antibody concentration, and the IC₅₀ for each antibody was determined by inhibitory curve fitting using GraphPad Prism software. The results of this assay demonstrate that the antibody containing an HC with the CDR3 insertion (encoded by SEQ ID NO: 30) exhibited an IC₅₀ that was 6-fold more potent than an antibody containing the same HC but which lacks the CDR3 mutation (encoded by SEQ ID NO: 29).

The results of this example demonstrate that the inventive method can be used to generate insertion mutations in an antibody-encoding nucleic acid sequence that increase the affinity of the antibody.

### Example 3

This example describes the presence, identity, and frequency of AID-mediated insertion and deletion mutations induced in antibody-encoding and non-antibody-encoding nucleic acid sequences.

HEK-293 c18 cell lines w^{e}re transfected with a plasmid containing a nucleic acid sequence encoding AID (SEQ ID NO: 15) alone, or with (i) a plasmid containing a nucleic acid sequence encoding a heavy chain polypeptide (HC) of an antibody to hen egg-white lysozyme (SEQ ID NO: 36) or (ii) a plasmid containing a nucleic acid sequence encoding an HC of an antibody to human nerve growth factor (NGF) (SEQ ID NO: 37). Specifically, a T75 culture flask was seeded with 3×10⁶ HEK293 c18 cells in 10 mL Dulbecco's Modified Eagle's Medium (DMEM) containing 10% FBS (Life Technologies, Carlsbad, Calif.). Plasmids were transfected using 500 μL OptiMEM (Life Technologies, Carlsbad, Calif.) and 20 μL HD-Fugene (Roche Diagnostics Corporation, Indianapolis, Ind.). Three da_{ys} post-transfection, cell growth medium was exchanged with 10 ml DMEM containing 10% FBS, 50 μg/ml GENETICIN™ aminoglycoside antibiotic (Life Technologies, Carlsbad, Calif.), 10 μl/mL Antibiotic-Antimycotic Solution, 1.5 μg/ml puromycin, 15 μg/ml blasticidin, and/or 350 μg/mL hygromycin (all from Life Technologies), and the cells were incubated for approximately four weeks with periodic reseeding and exchange of the cell culture medium. Following stable selection, HEK293-c18 cells were cultured in DMEM high glucose (Life Technologies, Carlsbad, Calif.) supplemented with 10% FBS, 250 μg/mL GENETICIN™ aminoglycoside antibiotic (Life Technologies, Carlsbad, Calif.), and 1× anti-anti (Life Technologies, Carlsbad, Calif.) at 37° C., 5% CO₂. Once cells were transfected with episomal plasmid vectors the following selection agents were added: hygromycin at 175 μg/mL for the light chain plasmid, puromycin at 0.75 μg/mL for the heavy chain plasmid, and/or blasticidin at 10 μg/mL for the plasmid encoding AID. Cells were maintained in a 6-well plate and split every two days over the course of the experiment using trypsin 0.25% EDTA. At the appropriate time points (from 28 to 75 days), cells were pelleted and lysed, and either the plasmid encoding the HC or AID was PCR amplified from the lysate, cloned, and submitted for bacterial colony sequencing.

Sequences were analyzed using LASEGENE™ SeqMan™ software (DNASTAR, Madison, Wis.) and aligned to the parental template. Poor sequence data was excluded, and good sequence data was trimmed to the variable region of the HC and 559 nucleotides from the ATG start codon for AID. The identity and number of mutations from the starting template were compiled using SeqMan's SNP detection. The resulting mutation data and the AID mutation rate were used to determine the spectrum of AID mutations induced.

In cells transfected only with the plasmid encoding AID, a total of 4,526 sequences were obtained following stable selection for time points ranging from 28 to 75 days. Insertion and deletion mutations were identified in the nucleic acid sequence encoding the AID protein (SEQ ID NO: 15), thereby indicating that AID acted as its own substrate. These insertion and deletion mutations are set forth in Table 1. The insertion and deletion mutations were found to initiate almost exclusively at G or C nucleotides. Twenty insertion or deletion mutations were observed among the 942 sequences collected, which is equivalent to a mutation frequency of 2.1%. The mutations ranged in length from deletions of 59 nucleotides to insertions of 21 nucleotides. Insertion and deletion mutations appeared to be localized to specific regions, even in the absence of functional selection (e.g., nucleotide positions 341, 344, 345, 348), thereby suggesting that local sequence features direct and support the formation of insertion and deletion mutations following deamination by AID. These local sequence features may be manipulated to effect the frequency, position, and characteristics of the insertion and deletion mutations observed.

TABLE 1

| Nucleotide | | |
|---|---|---|
| Position in | | |
| SEQ ID NO: 15 | Nucleic Acid | Nucleic Acid |
| of Insertion/ | Sequence | Sequence |
| Deletion | Before Mutation | After Mutation |
| 64 | ATGAAGCAGAGAGA | G |
| | GTTTCTCTACCACTTC | |
| | A | |
| | (SEQ ID NO: 39) | |
| 71 | AGAGAA | — |
| 71 | AGAGAA | — |
| 237 | CT | TC |
| 307 | — | C |
| 341 | TCAGGGGGTATCCC | — |
| | (SEQ ID NO: 40) | |
| 344 | G | — |
| 345 | GGGGTATCCCAATCT | — |
| | CTCCCTCCGCATATTC | |
| | GCCGCCCGACTCTAT | |
| | TTTTGTGAGGACA | |
| | (SEQ ID NO: 41) | |
| 348 | G | — |
| 348 | G | — |
| 348 | G | — |
| 399 | — | CCCGACTCTATTT |
| | | TTGTGA |
| | | (SEQ ID NO: 42) |
| 409 | — | TATTTTTGTGAGG |
| | | ACAGGAAA |
| | | (SEQ ID NO: 43) |
| 409 | — | TATTTTTGTGAGG |
| | | ACAGGAAA |
| | | (SEQ ID NO: 44) |
| 445 | — | CCTCGACCGGGCC |
| | | (SEQ ID NO: 45) |
| 449 | — | A |
| 555 | — | TTCAAAGCCT |
| | | (SEQ ID NO: 46) |
| 556 | G | — |
| 556 | G | — |
| 576 | T | — |

In cells transfected with the plasmid encoding AID and the plasmid encoding the HC of the hen egg-white lysozyme antibody, 1,623 sequences were obtained after stable co-expression for 28 days in the absence of functional selection. Insertion and deletion mutations were found to initiate almost exclusively at G or C nucleotides. 10 insertion or deletion mutations were observed among the 1,623 sequences collected, which is equivalent to a mutation frequency of 0.6%. The mutations ranged in length from deletions of 7 nucleotides to insertions of 15 nucleotides. The specific insertion and deletion mutations are set forth in Table 2.

TABLE 2

| Nucleotide | | |
|---|---|---|
| Position in | Nucleic | |
| SEQ ID NO: 36 | Acid | |
| of Insertion/ | Sequence | Nucleic Acid |
| Deletion | Before | Sequence |
| Mutation | Mutation | After Mutation |
| 18 | CTGCG | |
| 30 | G | — |
| 136 | — | TCACCTGTT |
| | | CTGTCA |
| | | (SEQ ID NO: 47) |
| 187 | G | — |
| 210 | G | — |
| 219 | CTACAGT | |
| 272 | C | — |
| 362 | — | CAAACTGG |
| | | GACGG |
| | | (SEQ ID NO: 48) |
| 419 | C | — |
| 419 | C | — |

In cells transfected with the plasmid encoding AID and the plasmid encoding the HC of an NGF antibody, 1,961 sequences were obtained after stable co-expression for 75 days in the absence of functional selection. Insertion and deletion mutations were found to initiate almost exclusively at G or C nucleotides. 12 insertion or deletion mutations were observed among the 1,961 sequences collected, which is equivalent to a mutation frequency of 0.61%. The mutations ranged in length from deletions of 11 nucleotides to insertions of 1 nucleotide. There did appear to be a spatial bias to insertion and deletion mutations, with 7 of the 12 insertion/deletion mutations located within 8 nucleotides of each other (e.g., nucleotides 413-421). The specific insertion and deletion mutations are set forth in Table 3.

TABLE 3

| Nucleotide | | |
|---|---|---|
| Position in | | Nucleic |
| SEQ ID NO: 37 | | Acid |
| of Insertion/ | Nucleic Acid | Sequence |
| Deletion | Sequence | After |
| Mutation | Before Mutation | Mutation |
| 56 | C | — |
| 124 | AAGGCTTCTGG | — |
| | (SEQ ID NO: 49) | |
| 176 | C | — |
| 368 | C | — |
| 368 | C | — |
| 413 | G | — |
| 416 | G | — |
| 416 | G | — |
| 416 | G | — |
| 416 | G | — |
| 420 | — | T |
| 421 | — | A |

The results of this example demonstrate that insertion and deletion mutations can be introduced into antibody- and non-antibody-encoding nucleic acid sequences in accordance with the inventive method.

### Example 4

This example describes the characteristics of insertion and deletion mutations induced in a nucleic acid sequence encoding an anti-MS2 antibody heavy chain polypeptide in accordance with the inventive method.

A plasmid containing a nucleic acid sequence encoding a anti-MS2 antibody heavy chain polypeptide (HC) (SEQ ID NO: 38) and a plasmid containing a nucleic acid encoding a corresponding light chain polypeptide (LC) were co-expressed with a plasmid containing a nucleic acid sequence encoding AID (SEQ ID NO: 15) in a HEK-293 c18 cell line.

The HC and LC sequences were affinity matured in a single round of FACS selection. Specifically, 50 million cells were pelleted at 100×g and resuspended in 100 mL PBS/1% BSA plus DyLight 649-labeled WFP-IL17a, and incubated at 4° C. or 25° C. for 1 to 2 hours. Cells were spun down and resuspended in 5 mL PBS/1% BSA with a 1:500 dilution of PE-labeled anti-human IgG (Rockland immunochemicals, Gilbertsville, Pa.) for 30 minutes at 4° C. or 25° C. Following a final pelleting, cells were resuspended in 2 mL PBS/1% BSA/0.1% DAPI. Flow cytometry was effected using a BD Influx™ cell sorter (BD Biosciences, Franklin Lakes, N.J.) at a flow rate of approximately 10,000 cells/sec, which was set to capture approximately 0.5% of the cell population.

Pre and post-FACS sort populations were isolated for high-throughput sequencing. HEK293 c-18 cell pellets (5×10⁶ cells) were lysed in sterile DNAse-free water at 95° C. for 15 minutes. 454 Amplicon fusion primers using Lib-A chemistry were designed according to 454 Sequencing Technical Bulletin #013-2009 (Roche Diagnostics, Indianapolis, Ind.). PCR amplification was run on the cell lysates using 5 units of Accuprime PFX (Life Technologies, Carlsbad, Calif.) and 20 μM of each primer. An initial denaturation step of 95° C. for 7 minutes was followed by 30 cycles of 95° C. for 25 seconds, 59° C. for 30 seconds, 68° C. for 45 seconds, and a final extension step of 68° C. for 7 minutes. Two reactions for each sample were (i) gel purified using a Zymo Research Zymoclean™ Gel DNA Recovery kit (Zymo Research Corp., Irvine, Calif.), (ii) combined, and (iii) eluted in 25 μl sterile DNAse-free water. Samples were further purified using Agencourt AMPure XP beads (Beckman Genomics, Danvers, Mass.), diluted to 10 ng/μl, and sequenced at 454 Life Sciences (Roche Diagnostics, Indianapolis, Ind.; GS FLX Titanium sequencing).

For detection of insertion and deletion mutations and analysis using high-throughput sequencing, two procedures were employed: (1) variant detection and noise screening followed by (2) haplotype identification on the deep sequencing data preprocessed with the Roche 454's default amplicon data processing pipeline (www.my454.com). Preprocessed reads in the format of sff files were mapped to the designated reference antibody sequence(s) with GSMapper from Roche 454 (www.my454.com). SNPs and insertion/deletion mutations were called from the pairwise alignments generated by GSMapper using VarScan (Koboldt et al., Bioinformatics, 25(17): 2283-2285 (epublished on Jun. 19, 2009). Instead of using the default noise filtering of VarScan, which was designed for human genome re-sequencing, a customized noise filtering based on recent reported 454 sequencing error models was employed (Gilles et al., BMC Genomics, 12: 245 (2011)). Briefly, the significance of a called variant (SNPs and insertion/deletion mutations) was computed following the Poisson distribution as:

$$P = {\sum }_{k = 0}^{m} p ( k ; λ ) = {\sum }_{k = 0}^{m} \frac{{λ}^{k} {e}^{- λ}}{k !}$$

Where m=the observed frequency of the variant, and λ=the expected variant frequency based on the employed sequencing error model. Variants with significance lower than a designated cutoff were discarded. Also excluded from consideration were sequences where insertion or deletion mutations, including single nucleotide polymorphisms (SNP), were observed close to the end or beginning of the sequencing run. In addition, sequences where insertion or deletion mutations occurred in a homopolymer run (i.e., a string of identical nucleotides greater than 3 nucleotides) were excluded from consideration. Based on the noise-screened variants, haplotypes were identified by examining the variants called on each reads. Haplotypes with limited supporting reads were treated as noise. In recognition of the stepwise accumulation of mutations, the association between haplotypes was characterized by their shared mutations and supporting. Haplotypes with strong read supporting (i.e., high frequency) and their association with other haplotypes were utilized to explore antibody affinity maturation, which lead to better affinity prediction than exploring individual variants.

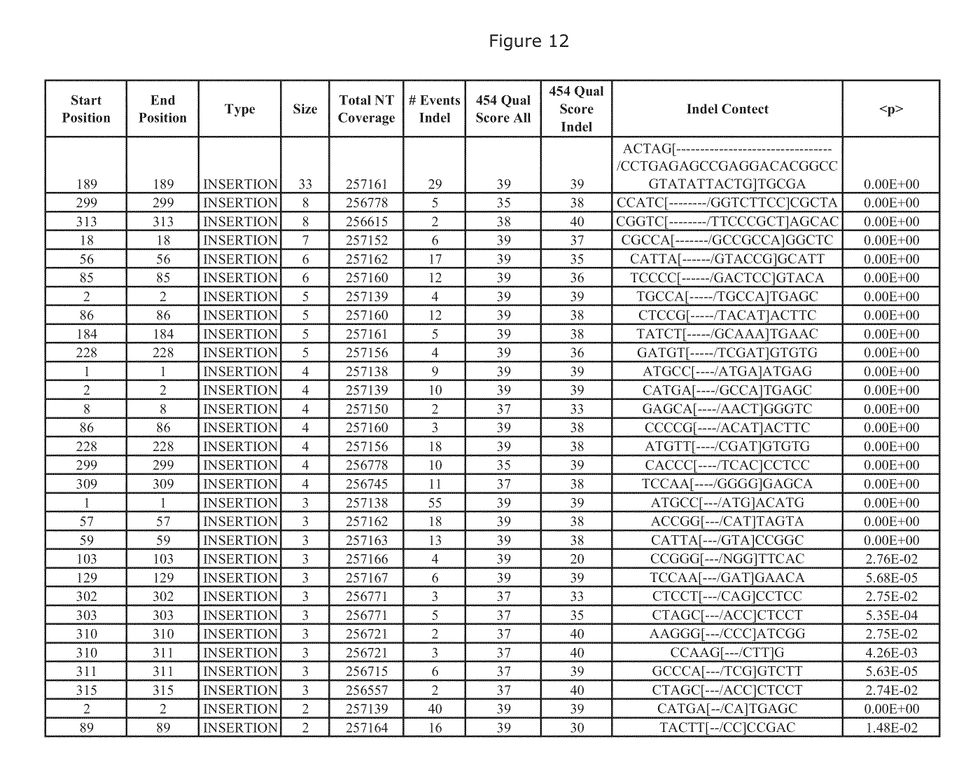

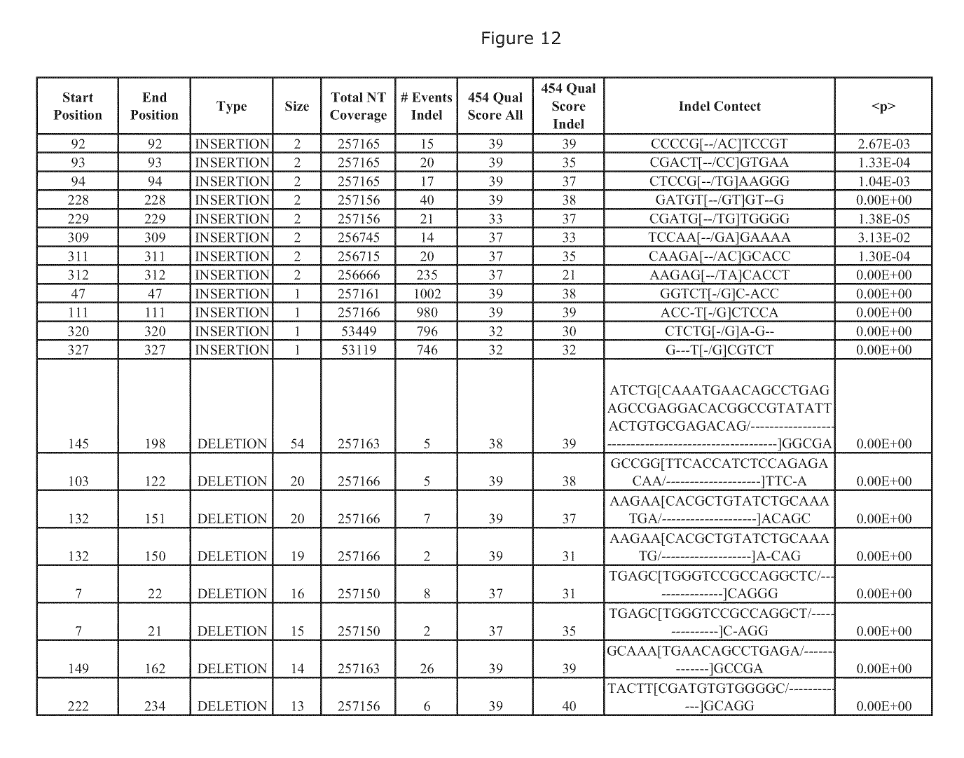

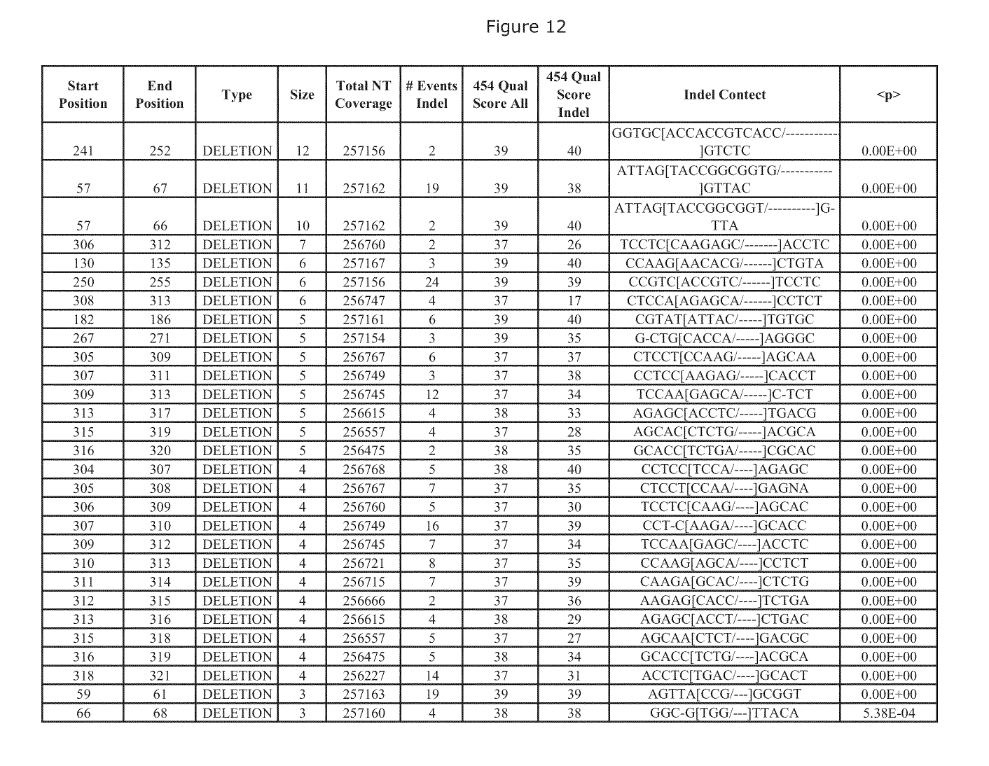

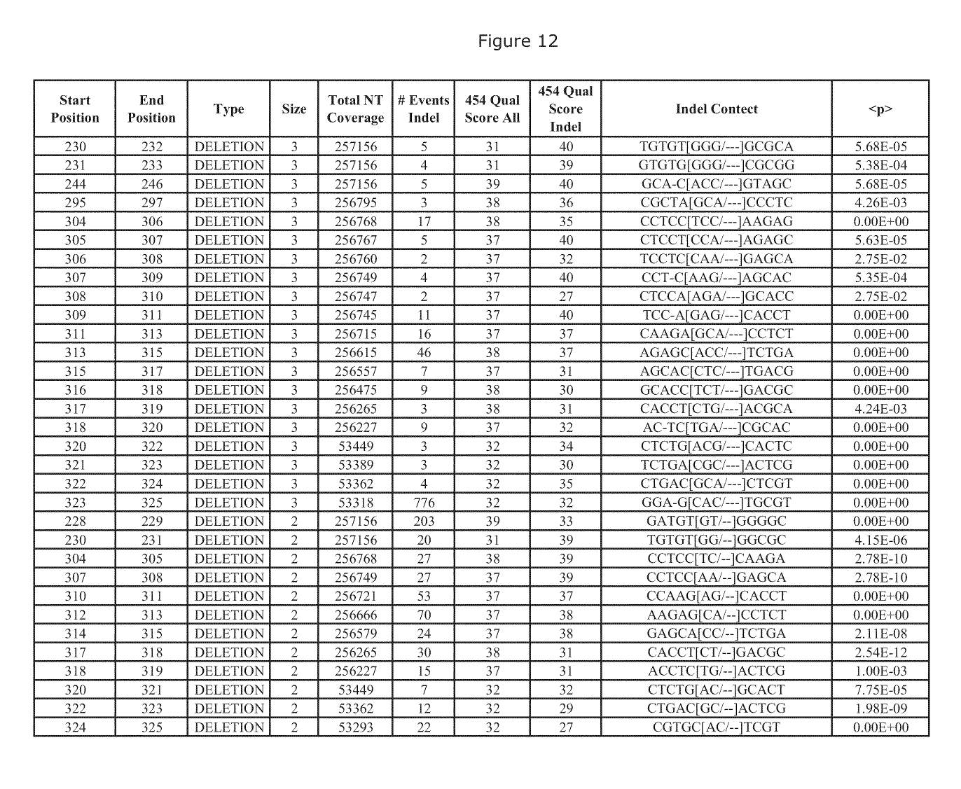

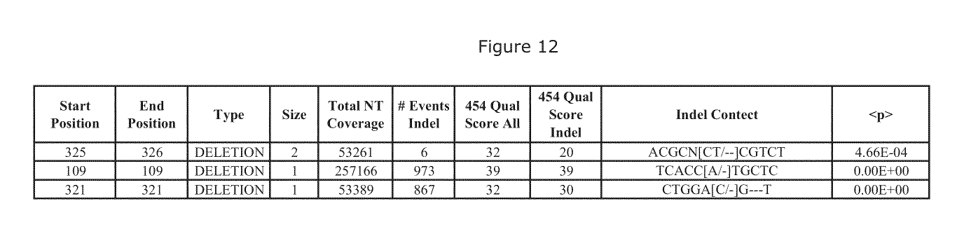

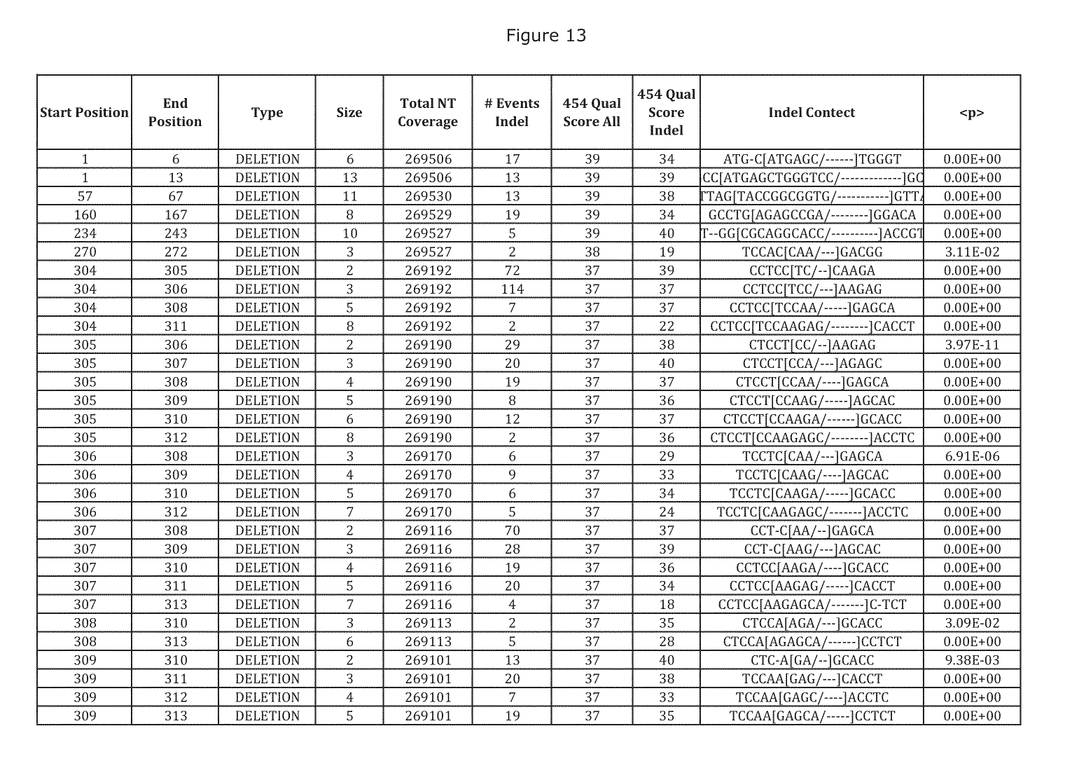

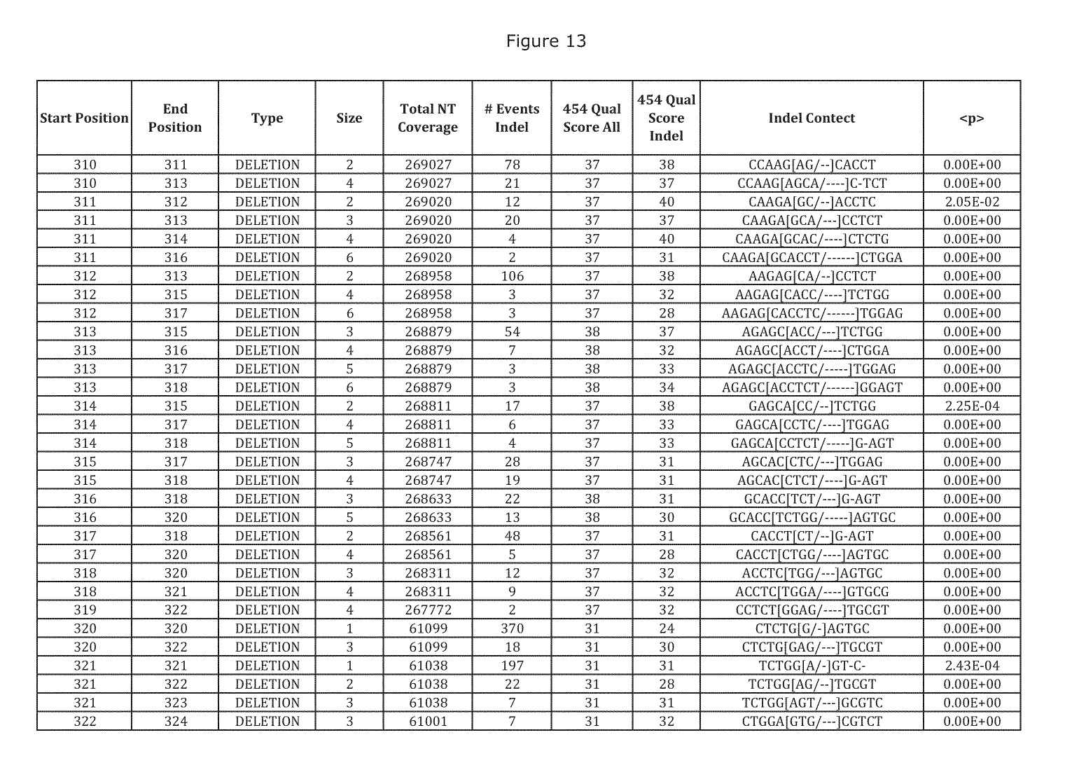

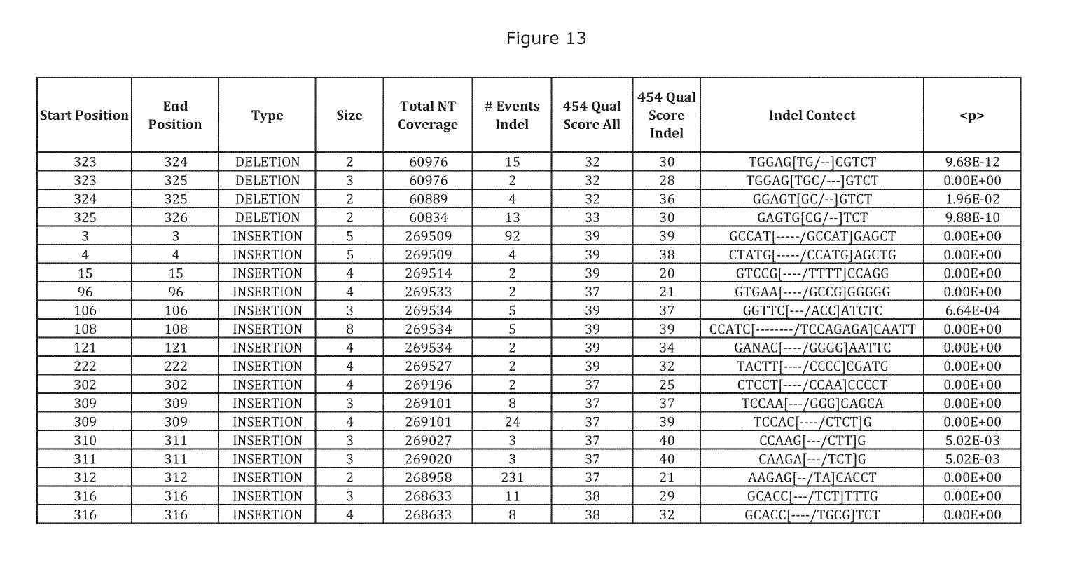

The insertion and deletion mutations detected in the pre- and post-FACS sort populations of nucleic acid sequences from the AID-expressing cells are shown in FIGS. 12 and 13, respectively.

The results of this example demonstrate that the inventive method can be used to generate insertion mutations and deletion mutations in an antibody-encoding nucleic acid sequence, and that high-throughput sequencing can identify insertion and deletion mutations that may impact antibody binding or function.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for introducing one or more insertion mutations and/or one or more deletion mutations in a nucleic acid sequence encoding a polypeptide, which method comprises:
(a) providing a cell in vitro that expresses or can be induced to express Activation Induced Cytidine Deaminase (AID),
(b) contacting the cell in vitro with a nucleic acid sequence that encodes a polypeptide, and
(c) expressing AID in the cell, whereupon one or more insertion mutations and/or one or more deletion mutations are introduced in the nucleic acid sequence encoding the polypeptide.

2. The method of claim 1, wherein the nucleic acid sequence encodes an immunoglobulin heavy chain polypeptide.

3. The method of claim 2, wherein one or more insertion mutations are introduced in the nucleic acid sequence encoding the immunoglobulin heavy chain polypeptide.

4. The method of claim 3, wherein the one or more insertion mutations result in the insertion of one to eight amino acid residues into the immunoglobulin heavy chain polypeptide.

5. The method of claim 2, wherein one or more deletion mutations are introduced in the nucleic acid sequence encoding the immunoglobulin heavy chain polypeptide.

6. The method of claim 5, wherein the one or more deletion mutations result in the deletion of one to eight amino acid residues from the immunoglobulin heavy chain polypeptide.

7. The method of claim 2, wherein the one or more insertion mutations and/or the one or more deletion mutations occur in a complementarity determining region (CDR) of the immunoglobulin heavy chain polypeptide.

8. The method of claim 7, wherein the one or more insertion mutations and/or the one or more deletion mutations occur in CDR3 of the immunoglobulin heavy chain polypeptide.

9. An isolated or purified nucleic acid sequence which encodes an immunoglobulin heavy chain polypeptide prepared by the method of claim 2.

10. The method of claim 1, wherein the nucleic acid sequence encodes an immunoglobulin light chain polypeptide.

11. The method of claim 10, wherein one or more insertion mutations are introduced in the nucleic acid sequence encoding the immunoglobulin light chain polypeptide.

12. The method of claim 11, wherein the one or more insertion mutations result in the insertion of one to eight amino acid residues into the immunoglobulin light chain polypeptide.

13. The method of claim 10, wherein one or more deletion mutations are introduced in the nucleic acid sequence encoding the immunoglobulin light chain polypeptide.

14. The method of claim 13, wherein the one or more deletion mutations result in the deletion of one to eight amino acid residues from the immunoglobulin light chain polypeptide.

15. The method of claim 10, wherein the one or more insertion mutations and/or the one or more deletion mutations occurs in a complementarity determining region (CDR) of the immunoglobulin light chain polypeptide.

16. The method of claim 15, wherein the one or more insertion mutations and/or the one more deletion mutations occur in CDR3 of the immunoglobulin light chain polypeptide.

17. An isolated or purified nucleic acid sequence which encodes an immunoglobulin light chain polypeptide prepared by the method of claim 10.

18. A method of producing an immunoglobulin, which method comprises:
(a) providing a cell in vitro that expresses or can be induced to express AID,
(b) contacting the cell in vitro with the nucleic acid sequence encoding an immunoglobulin heavy chain polypeptide of claim 9 and the nucleic acid sequence encoding an immunoglobulin light chain polypeptide of claim 17, and
(c) expressing AID in the cell, whereupon an immunoglobulin comprising a heavy chain polypeptide and a light chain polypeptide is produced in the cell.

19. The method of claim 18, wherein the cell is a eukaryotic cell.

20. The method of claim 19, wherein the cell is a B-cell.

21. The method of claim 19, wherein the cell is an HEK-293 cell.

22. The method of claim 1, wherein the cell is a eukaryotic cell.
